SEQUENCE LISTING (15 sequences)

SEQ ID NO: 1 - DNA, 2847 bp, Mus musculus
atgaaagggtccctcctgctgctgctggtgtcaaacctgctcctgtgccagagcgggtccggagccggggcccacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcttgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaacccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctaaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttga

SEQ ID NO: 2 - protein, 948 aa, Mus musculus
MKGSLLLLLVSNLLLCQSGSGAGAHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGLWIDMNEPSNFIRGSEDGCPNNELENPPYVPGVVGGTLQAATICASSHQFLSTHYNLHNLYGLTEPIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 3 - DNA, 43 bp, Artificial Sequence
  Synthetic Primer
gcgataggtaccgccatgggagtgaggcacccgccctgctccc

SEQ ID NO: 4 - DNA, 43 bp, Artificial Sequence
  Synthetic Primer
gcgatactcgagtcaacaccagctgacgagaaactgctctccc

SEQ ID NO: 5 - protein, 28 aa, Homo sapiens
MGVRHPPCSHRLLAVCALVSLATAALLG

SEQ ID NO: 6 - protein, 35 aa, Homo sapiens
MGPRGAASLPRGPGPRRLLLPVVPLLLLLLLAPPG

SEQ ID NO: 7 - protein, 18 aa, Homo sapiens
MKGSLLLLLVSNLLLCQS

SEQ ID NO: 8 - protein, 6 aa, Homo sapiens
GSGAGA

SEQ ID NO: 9 - DNA, 2847 bp, Homo sapiens; identical to SEQ ID NO: 1

SEQ ID NO: 10 - protein, 948 aa, Homo sapiens
MKGSLLLLLVSNLLLCQSGSGAGAHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLPTPGANLYGSHPFYLALEDGSPLMLSTSWTRITLWNRDLAGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGLWIDMNEPSNFIRGSEDGCPNNELENPPYVPGVVGGTLQAATICASSHQFLSTHYNLHNLYGLTEPIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 11 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 12 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFNCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGLWIDMNEPSNFIRGSEDGCPNNELENPPYVPGVVGGTLQAATICASSHQFLSTHYNLHNLYGLTEPIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 13 - DNA, 2859 bp, Homo sapiens
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 14 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGLWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 15 - protein, 952 aa, Homo sapiens [sequence omitted]

(19) United States
(12) Patent Application Publication
(10) Pub. No.: US 2009/0191178 A1
(43) Pub. Date: Jul. 30, 2009

(54) Manufacture of Highly Phosphorylated Lysosomal Enzymes and Uses Thereof

(75) Inventors: Todd C. Zankel, San Francisco, CA (US); Christopher M. Starr, Sonoma, CA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: BIOMARIN PHARMACEUTICAL INC., Novato, CA (US)

(21) Appl. No.: 12/182,818

(22) Filed: Jul. 30, 2008

Related U.S. Application Data
(63) Continuation-in-part of application No. 10/588,425, filed on Jun. 6, 2007.
(60) Provisional application No. 60/542,586, filed on Feb. 6, 2004.

(51) Int. Cl. A61K 38/47; C12N 9/24; C12P 21/04; C12N 5/06

(52) U.S. Cl. 424/94.61; 435/200; 435/69.1; 435/358

(57) ABSTRACT

This invention provides compositions of highly phosphorylated lysosomal enzymes, their pharmaceutical compositions, methods of producing and purifying such lysosomal enzymes and compositions and their use in the diagnosis, prophylaxis, or treatment of diseases and conditions, including particularly lysosomal storage diseases.

### CROSS-REFERENCE TO RELATED APPLICATIONS

This application is a continuation-in-part of U.S. application Ser. No. 10/588,425, filed Jun. 6, 2007, which is the National Stage of International Application No. PCT/US2005/004345, filed Feb. 7, 2005, which claims the benefit of and priority to U.S. Provisional Application No. 60/542,586, filed Feb. 6, 2004, the disclosures of which are herein incorporated by reference in their entirety.

### FIELD OF THE INVENTION

The present invention relates to the technical fields of cellular and molecular biology and medicine, particularly to the manufacture of highly phosphorylated lysosomal enzymes and their use in the management of lysosomal storage diseases.

### BACKGROUND OF THE INVENTION

Lysosomal storage diseases (LSDs) result from the deficiency of specific lysosomal enzymes within the cell that are essential for the degradation of cellular waste in the lysosome. A deficiency of such lysosomal enzymes leads to accumulation of undegraded “storage material” within the lysosome, which causes swelling and malfunction of the lysosomes, and ultimately cellular and tissue damage. A large number of lysosomal enzymes have been identified and correlated with their related diseases. Once a missing enzyme has been identified, treatment can be reduced to the sole problem of efficiently delivering replacement enzyme to the affected tissues of patients.

One way to treat lysosomal storage diseases is by intravenous enzyme replacement therapy (ERT) (Kakkis, Expert Opin. Investig. Drugs 11(5):675-685, 2002). ERT takes advantage of the vasculature to carry enzyme from a single site of administration to most tissues. Once the enzyme has been widely distributed, it must be taken up into cells. The basis for uptake into cells is found in a unique feature of lysosomal enzymes, which constitute a separate class of glycoproteins defined by phosphate at the 6-position of terminal mannose residues. Mannose-6-phosphate (M6P) is bound with high affinity and specificity by a receptor found on the surface of most cells (Munier-Lehmann et al., Biochem. Soc. Trans. 24(1):133-136, 1996; Marnell et al., J. Cell. Biol. 99(6): 1907-1916, 1984). The mannose-6-phosphate receptor (MPR) directs uptake of enzyme from blood to tissue and then mediates intracellular routing to the lysosome.

The therapeutic effectiveness of a lysosomal enzyme preparation depends crucially on the level of mannose 6-phosphate in that preparation. Phosphate is added to the glycoprotein by a post-translational modification in the endoplasmic reticulum and early Golgi. Folded lysosomal enzymes display a unique tertiary determinant that is recognized by an oligosaccharide modification enzyme. The determinant is composed of a set of specifically spaced lysines and is found on most lysosomal enzymes despite absence of primary sequence homology. The modification enzyme, UDP-GlcNAc phosphotransferase, binds to the protein determinant and adds GlcNAc-1-phosphate to the 6-position of terminal mannose residues on oligosaccharides proximate to the binding site. A second enzyme, phosphodiester α-GlcNAcase, then cleaves the GlcNAc-phosphate bond to remove N-acetylglucosamine to give a mannose 6-phosphate terminal oligosaccharide. The purpose of the mannose 6-phosphate modification is to divert lysosomal enzymes from the secretory pathway to the lysosomal pathway within the cell. Phosphate-bearing enzyme is bound by the MPR in the trans Golgi and routed to the lysosome instead of the cell surface.

Large-scale production of lysosomal enzymes involves expression in mammalian cell lines. The goal is the predominant secretion of recombinant enzyme into the surrounding growth medium for harvest and processing downstream. In an ideal system for the large-scale production of lysosomal enzymes, enzyme would be efficiently phosphorylated and then directed primarily toward the cell surface (secretion) rather than primarily to the lysosome. As described above, this proportionation of phosphorylated enzymes is the exact opposite of what occurs in normal cells. Manufacturing lines often used for lysosomal enzyme production focus on maximizing the level of mannose 6-phosphate per mole of enzyme and are characterized by low specific productivity. In vitro attempts at producing lysosomal enzymes containing high levels of mannose-6 phosphate moieties have resulted in mixed success (Canfield, U.S. Pat. No. 6,537,785). The in vitro enzyme exhibits high levels of mannose-6-phosphate, as well as high levels of unmodified terminal mannose. Competition between the mannose 6-phosphate and mannose receptors for enzyme results in the necessity for high doses of enzyme for effectiveness, and could lead to greater immunogenicity to the detriment of the subject being treated.

Thus, there exists a need in the art for an efficient and productive system for the large-scale manufacture of therapeutically effective lysosomal enzymes for management of lysosomal storage disorders.

### SUMMARY OF INVENTION

The present invention relates to the discovery that a CHO-K1 derivative, designated G71, which is defective in endosomal acidification, produces high yields of phosphorylated, recombinant enzyme by preventing loss of material to the lysosomal compartment of the manufacturing cell line itself. Such enzymes also preferably have a low level of unphosphorylated high-mannose oligosaccharides. In one embodiment, the invention provides an END3 complementation group cell line that overexpresses recombinant lysosomal enzymes, resulting in high yields of highly phosphorylated enzyme. Exemplary cell lines are G71 or a derivative thereof that retains the desired property of G71, i.e., the ability to produce high yields of highly phosphorylated recombinant enzyme preferably with a low level of unphosphorylated high mannose oligosaccharides. This application of an END3 complementation group modified CHO-K1 line would be especially useful for the manufacture of lysosomal enzymes to be used for management of lysosomal storage diseases by enzyme replacement therapy (ERT). In one embodiment, the lysosomal enzyme can be human acid alpha-glucosidase (rhGAA).

In one aspect, the present invention features a novel method of producing highly phosphorylated lysosomal enzymes in amounts that enable their therapeutic use. In a broad embodiment, the method comprises the step of transfecting a cDNA encoding for all or part of the lysosomal enzyme into a cell suitable for the expression thereof. In some embodiments, a cDNA encoding for a full-length lysosomal enzyme is used, whereas in other embodiments a cDNA encoding for a biologically active fragment, variant, derivative or mutant thereof may be used. In other preferred embodiments, an expression vector is used to transfer the cDNA into a suitable cell line for expression thereof. In a preferred embodiment, the method comprises the step of producing highly phosphorylated lysosomal enzymes from cell lines with defects in endosomal trafficking. In a particularly preferred embodiment, the method comprises the step of producing highly phosphorylated recombinant lysosomal enzymes from the END3 complementation group CHO cell line G71. In one embodiment, the lysosomal enzyme can be human acid alpha-glucosidase (rhGAA).

In a preferred embodiment, the invention provides a method for producing a recombinant human acid alpha-glucosidase (rhGAA) enzyme or variant thereof, or derivative of the rhGAA enzyme or variant thereof, which has a high level of phosphorylation and a low level of unphosphorylated high-mannose oligosaccharides, exceeding at least about 0.7 moles bis-phosphorylated oligomannose chains per mole of rhGAA enzyme or variant thereof, comprising the steps of: (a) culturing Chinese Hamster Ovary (CHO)-derived END3 complementation group cells, preferably G71 cells or a derivative thereof; (b) preparing a mammalian expression vector encoding the rhGAA enzyme or variant thereof suitable for the END3 complementation group cells; (c) transfecting the END3 complementation group cells with the expression vector; (d) selecting and cloning a END3 complementation group cell transfectant; and (e) optimizing cell culture process methods for manufacturing the END3 complementation group cell transfectant. In a more preferred embodiment, the rhGAA enzyme comprises an amino acid sequence selected from the group consisting of SEQ ID NO:11 (GAA1-M), SEQ ID NO:12 (GAA2), SEQ ID NO:14 (GAA1-L) and SEQ ID NO:15 (GAA4). In a most preferred embodiment, the rhGAA enzyme comprises the amino acid sequence of SEQ ID NO:11 (GAA1-M). In another preferred embodiment, the rhGAA enzyme has high levels of phosphorylation (greater than about 0.7 moles oligomannose bis-phosphate per mole of enzyme, preferably greater than about 0.85 moles oligomannose bis-phosphate per mole of enzyme, and more preferably greater than about 1.0 mole oligomannose bis-phosphate per mole of enzyme) and low levels of high-mannose oligosaccharide.

In a second aspect, the present invention provides an endosomal acidification-deficient cell line characterized by its ability to produce lysosomal enzymes in amounts that enable use of the enzyme therapeutically. In preferred embodiments, the invention provides CHO-K1-derived END3 complementation group cell lines, designated G71 and derivatives thereof, that are capable of producing high yields of highly phosphorylated lysosomal enzymes, thereby enabling the large scale production of therapeutic lysosomal enzymes. In most preferred embodiments, the cell line expresses and secretes recombinant lysosomal enzymes in amounts of at least about 0.25 picogram/cell/day, preferably at least about 0.5 picogram/cell/day, more preferably at least about 1.0 picogram/cell/day, or more.

An END3 complementation group cell line is any modified CHO cell line that retains the properties of an END3 complementation group cell, such as defective endosomal acidification. In one embodiment, the END3 complementation group CHO cell line is derived from G71 or a derivative thereof and comprises an expression vector for recombinant human acid alpha-glucosidase (rhGAA). In a preferred embodiment, the END3 complementation group CHO cell line expresses and secretes rhGAA. In another preferred embodiment, the END3 complementation group cell line is selected from the group consisting of 1B8, C3, D7 and F5. In a more preferred embodiment, the END3 complementation group cell line is C3. In another preferred embodiment, the END3 complementation group cell line comprises G71 or a derivative thereof that is adapted to growth in suspension.

In a third aspect, the invention provides novel lysosomal enzymes produced in accordance with the methods of the present invention and thereby present in amounts that enable using the enzyme therapeutically. The enzymes may be full-length proteins, or fragments, variants, derivatives or mutants thereof. In some embodiments, the full-length protein, or fragment, variant, derivative or mutant thereof according to the invention may be modified as desired to enhance its stability or pharmacokinetic properties (e.g., PEGylation, mutagenesis, fusion, conjugation). In preferred embodiments, the enzyme is a human enzyme, a fragment of the human protein or enzyme having a biological activity of a native protein or enzyme, or a polypeptide that has substantial amino acid sequence homology with the human protein or enzyme. In some embodiments, the enzyme agent is a protein of human or mammalian sequence, origin or derivation. In other embodiments, the enzyme or protein is such that its deficiency causes a human disease such as Pompe disease (e.g. acid alpha-glucosidase). In other embodiments, the enzyme is selected for its beneficial effect.

The enzyme or protein can also be of human or mammalian sequence origin or derivation. In yet other embodiments of the invention, in each of its aspects, the enzyme or protein is identical in amino acid sequence to the corresponding portion of a human or mammalian polypeptide amino acid sequence. In other embodiments, the polypeptide moiety is the native protein from the human or mammal. In other embodiments, the polypeptide is substantially homologous (i.e., at least 80%, 85%, 90%, 95%, 96%, 97%, 98%, or 99% identical in amino acid sequence) over a length of at least 25, 50, 100, 150, or 200 amino acids, or the entire length of the polypeptide, to the native enzyme sequence of human or mammalian enzyme. In other embodiments, the subject to which the enzyme is to be administered is human.

In a preferred embodiment, the lysosomal enzyme of the invention is a recombinant human acid alpha-glucosidase (rhGAA) enzyme or variant thereof, or derivative of the rhGAA enzyme or variant thereof, which is produced in END3 complementation group CHO cells, preferably a G71 cell line or derivative thereof, and which has a high level of phosphorylation and a low level of unphosphorylated high-mannose oligosaccharides, exceeding at least about 0.7 moles bis-phosphorylated oligomannose chains per mole of rhGAA enzyme or variant thereof. In a more preferred embodiment, the rhGAA enzyme comprises an amino acid sequence selected from the group consisting of SEQ ID NO:11 (GAA1-M), SEQ ID NO:12 (GAA2), SEQ ID NO:14 (GAA1-L) and SEQ ID NO:15 (GAA4). In a most preferred embodiment, the rhGAA enzyme comprises the amino acid sequence of SEQ ID NO:11 (GAA1-M).

In another preferred embodiment, the lysosomal enzyme of the invention is a recombinant human acid alpha-glucosidase (rhGAA) enzyme or variant thereof, or derivative of said rhGAA enzyme or variant thereof, which has a high level of phosphorylation and a low level of unphosphorylated high-mannose oligosaccharides, exceeding at least about 0.7 moles bis-phosphorylated oligomannose chains per mole of rhGAA enzyme or variant thereof, which is produced by the methods of the invention. In a more preferred embodiment, the rhGAA enzyme comprises an amino acid sequence selected from the group consisting of SEQ ID NO:11 (GAA1-M), SEQ ID NO:12 (GAA2), SEQ ID NO:14 (GAA1-L) and SEQ ID NO:15 (GAA4). In a most preferred embodiment, the rhGAA enzyme comprises the amino acid sequence of SEQ ID NO:11 (GAA1-M).

In preferred embodiments, the enzyme is a recombinant human lysosomal enzyme produced by an endosomal acidification-deficient cell line. In more preferred embodiments, the recombinant human lysosomal enzyme has a high level of phosphorylated oligosaccharides, exceeding at least about 0.7 moles bis-phosphorylated oligomannose chains per mole of protein, preferably exceeding at least about 0.85 moles bis-phosphorylated oligomannose chains per mole of protein, and more preferably exceeding at least about 1.0 mole bis-phosphorylated oligomannose chains per mole of protein, and low level of unphosphorylated high-mannose oligosaccharides. In most preferred embodiments, the enzyme is a highly phosphorylated recombinant human acid alpha-glucosidase (rhGAA).

In a fourth aspect, the invention provides a method to purify the lysosomal enzymes produced by the methods of the present invention. In preferred embodiments, the invention provides a method for purifying a lysosomal enzyme comprising the steps of: (a) dia-filtering a cell culture harvest containing the lysosomal enzyme; (b) adjusting the pH of the dia-filtered harvest to induce precipitation of contaminating proteins; and (c) isolating the lysosomal enzyme by sequential chromatography on dye-ligand, anion exchange and hydrophobic resins. In other preferred embodiments, the invention provides a method for purifying a lysosomal enzyme comprising the steps of: (a) ultra-filtering a cell culture harvest containing the lysosomal enzyme; (b) adjusting the pH of the ultra-filtered harvest to induce precipitation of contaminating proteins; and (c) isolating the lysosomal enzyme by sequential chromatography on dye-ligand, anion exchange, cation exchange and hydrophobic resins. In some embodiments, the dye-ligand resin is Blue-Sepharose. In some embodiments, the anion exchange resin is Q-Sepharose. In some embodiments, the cation exchange resin is SE Hi-Cap. In some embodiments, the hydrophobic resin is Phenyl-Sepharose.

In a fifth aspect, the present invention provides a method of treating diseases caused all or in part by deficiency of lysosomal enzyme. In most preferred embodiments, the method comprises administering the therapeutic enzyme produced by the methods of the present invention, wherein the enzyme binds to an MPR receptor and is transported across the cell membrane, enters the cell and is delivered to the lysosomes within the cell. In one embodiment, the method comprises administering a therapeutic recombinant enzyme, or a biologically active fragment, variant, derivative or mutant thereof, alone or in combination with a pharmaceutically acceptable carrier. In other embodiments, this method features transfer of a nucleic acid sequence encoding the full-length lysosomal enzyme or a fragment, variant, derivative or mutant thereof into one or more of the host cells in vivo. Preferred embodiments include optimizing the dosage to the needs of the subjects to be treated, preferably mammals and most preferably humans, to most effectively ameliorate the disease symptoms.

Such therapeutic enzymes are particularly useful, for example, in the treatment of lysosomal storage diseases such as MPS I, MPS II, MPS III A, MPS III B, Metachromatic Leukodystrophy, Gaucher, Krabbe, Pompe, Neuronal Ceroid Lipofuscinosis CLN2 Batten disease, Niemann-Pick and Tay-Sachs disease wherein a lysosomal protein deficiency contributes to the disease state. In yet other embodiments, the invention also provides a pharmaceutical composition comprised of the deficient protein or enzyme causing a lysosomal storage disease.

In some embodiments, the lysosomal enzymes, compositions, and methods of the invention can be used to treat such lysosomal storage diseases as Aspartylglucosaminuria, Cholesterol ester storage disease/Wolman disease, Cystinosis, Danon disease, Fabry disease, Farber Lipogranulomatosis/Farber disease, Fucosidosis, Galactosialidosis types I/II, Gaucher disease types I/II/III Gaucher disease, Globoid cell leukodystrophy/Krabbe disease, Glycogen storage disease II/Pompe disease, GM1-Gangliosidosis types I/II/III, GM2-Gangliosidosis type I/Tay-Sachs disease, GM2-Gangliosidosis type II Sandhoff disease, GM2-Gangliosidosis, alpha-Mannosidosis types I/II, alpha-Mannosidosis, Metachromatic leukodystrophy, Mucolipidosis type I/Sialidosis types I/II Mucolipidosis types II/III I-cell disease, Mucolipidosis type IIIC pseudo-Hurler polydystrophy, Mucopolysaccharidosis type I, Mucopolysaccharidosis type II Hunter syndrome, Mucopolysaccharidosis type IIIA Sanfilippo syndrome, Mucopolysaccharidosis type IIIB Sanfilippo syndrome, Mucopolysaccharidosis type IIIC Sanfilippo syndrome, Mucopolysaccharidosis type IIID Sanfilippo syndrome, Mucopolysaccharidosis type IVA Morquio syndrome, Mucopolysaccharidosis type IVB Morquio syndrome, Mucopolysaccharidosis type VI, Mucopolysaccharidosis type VII Sly syndrome, Mucopolysaccharidosis type IX, Multiple sulfatase deficiency, Pompe, Neuronal Ceroid Lipofuscinosis CLN1 Batten disease, Neuronal Ceroid Lipofuscinosis CLN2 Batten disease, Niemann-Pick disease types A/B Niemann-Pick disease, Niemann-Pick disease type C1 Niemann-Pick disease, Niemann-Pick disease type C2 Niemann-Pick disease, Pycnodysostosis, Schindler disease types I/II Schindler disease, and Sialic acid storage disease. In particularly preferred embodiments, the lysosomal storage disease is MPS III, MLD, or GM1. In a most preferred embodiment, the lysosomal storage disease is Pompe.

In still another embodiment, the present invention provides for a method of enzyme replacement therapy by administering a therapeutically effective amount of a fusion or conjugate to a subject in need of the enzyme replacement therapy, wherein the cells of the patient have lysosomes which contain insufficient amounts of the enzyme to prevent or reduce damage to the cells, whereby sufficient amounts of the enzyme enter the lysosomes to prevent or reduce damage to the cells. The cells may be within or without the CNS or need not be set off from the blood by capillary walls whose endothelial cells are closely sealed to diffusion of an active agent by tight junctions.

In a particular embodiment, the invention provides lysosomal enzymes comprising an active agent having a biological activity which is reduced, deficient, or absent in the target lysosome and which is administered to the subject. Preferred active agents include, but are not limited to aspartylglucosaminidase, acid lipase, cysteine transporter, Lamp-2, alpha-galactosidase A, acid ceramidase, alpha-L-fucosidase, beta-hexosaminidase A, GM2-activator deficiency, alpha-D-mannosidase, beta-D-mannosidase, arylsulfatase A, saposin B, neuraminidase, alpha-N-acetylglucosaminidase phosphotransferase, phosphotransferase γ-subunit, alpha-L-iduronidase, iduronate-2-sulfatase, heparan-N-sulfatase, alpha-N-acetylglucosaminidase, acetylCoA:N-acetyltransferase, N-acetylglucosamine 6-sulfatase, galactose 6-sulfatase, alpha-galactosidase, N-acetylgalactosamine 4-sulfatase, hyaluronoglucosaminidase, palmitoyl protein thioesterase, tripeptidyl peptidase I, acid sphingomyelinase, cholesterol trafficking, cathepsin K, beta-galactosidase B. α-glucosidase, and sialic acid transporter. In a preferred embodiment, alpha-L-iduronidase, α-glucosidase or N-acetylgalactosamine 4-sulfatase is the enzyme. In a most preferred embodiment, α-glucosidase is the enzyme.

In a preferred embodiment, the invention provides a method of treating Pompe disease by administering a lysosomal enzyme of the invention useful to treat Pompe disease, wherein the lysosomal enzyme is a recombinant human acid alpha-glucosidase (rhGAA) enzyme or variant thereof, or derivative of said rhGAA enzyme or variant thereof, which is produced by END3 complementation group cells, preferably G71 cells or a derivative thereof, and which has a high level of phosphorylation and a low level of unphosphorylated high-mannose oligosaccharides, exceeding at least about 0.7 moles bis-phosphorylated oligomannose chains per mole of rhGAA enzyme or variant thereof. In a more preferred embodiment, the rhGAA enzyme comprises an amino acid sequence selected from the group consisting of SEQ ID NO:11 (GAA1-M), SEQ ID NO:12 (GAA2), SEQ ID NO:14 (GAA1-L) and SEQ ID NO:15 (GAA4). In a most preferred embodiment, the rhGAA enzyme comprises the amino acid sequence of SEQ ID NO:11 (GAA1-M). In another preferred embodiment, the rhGAA enzyme has high levels of phosphorylation (greater than about 0.7 moles oligomannose bis-phosphate per mole of enzyme, preferably greater than about 0.85 moles oligomannose bis-phosphate per mole of enzyme, and more preferably greater than about 1.0 mole oligomannose bis-phosphate per mole of enzyme) and low levels of high-mannose oligosaccharide.

Corresponding use of highly phosphorylated lysosomal enzymes of the invention, which are preferably produced by methods of the invention, in preparation of a medicament for the treatment of the diseases described above is also contemplated.

In a sixth aspect, the present invention provides pharmaceutical compositions comprising recombinant therapeutic lysosomal enzymes useful for treating a disease caused all or in part by the deficiency in such lysosomal enzyme. Such compositions may be suitable for administration by several routes such as intrathecal, parenteral, topical, intranasal, inhalational or oral administration. Within the scope of this aspect are embodiments featuring nucleic acid sequences encoding the full-length enzymes or fragments, variants, derivatives or mutants thereof, which may be administered in vivo into cells affected with a lysosomal enzyme deficiency.

The lysosomal enzyme of the invention may be formulated as a pharmaceutical composition for therapeutic use. In a preferred embodiment, the pharmaceutical composition comprises a recombinant human acid alpha-glucosidase (rhGAA) enzyme or variant thereof, or derivative of said rhGAA enzyme or variant thereof, wherein the rhGAA enzyme or variant thereof has at least about 0.7 bis-phosphorylated oligomannose chains per mole of rhGAA enzyme, which is produced comprising the steps of: (a) culturing Chinese Hamster Ovary (CHO)-derived END3 complementation group cells; (b) preparing a mammalian expression vector encoding the rhGAA enzyme or variant thereof suitable for the END3 complementation group cells; (c) transfecting the END3 complementation group cells with the expression vector; (d) selecting and cloning a END3 complementation group cell transfectant; and (e) optimizing cell culture process methods for manufacturing the END3 complementation group cell transfectant, wherein the END3 complementation group cell is a G71 cell line or a derivative thereof, and a pharmaceutically acceptable carrier, diluent or excipient. In a more preferred embodiment, the rhGAA enzyme comprises an amino acid sequence selected from the group consisting of SEQ ID NO:11 (GAA1-M), SEQ ID NO:12 (GAA2), SEQ ID NO:14 (GAA1-L) and SEQ ID NO:15 (GAA4). In an even more preferred embodiment, the rhGAA comprises the amino acid sequence of SEQ ID NO:11 (GAA1-M). In another more preferred embodiment, the rhGAA enzyme has at least about 0.85 bis-phosphorylated oligomannose chains per mole of rhGAA enzyme. In an even more preferred embodiment, the rhGAA enzyme has at least about 1.0 bis-phosphorylated oligomannose chains per mole of rhGAA enzyme.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

FIG. 1 describes the nucleic acid sequences of the primers used to amplify human acid alpha-glucosidase (hGAA) from human liver mRNA by high-stringency PCR (SEQ ID NOs: 3 and 4).

FIG. 2 describes (A) the CINt vector and (B) the pBMP14 vector.

FIG. 3 describes the nucleotide (top) and amino acid (bottom) sequences (SEQ ID NOs: 1 and 2, respectively) of human acid alpha-glucosidase (hGAA) inserted into the CINt vector.

FIG. 4 shows an amino acid sequence alignment of human acid alpha-glucosidase isoform 1 (GAA1, top, SEQ ID NO:11) and 2 (GAA2, bottom, SEQ ID NO:12).

FIG. 5 describes the nucleotide sequence of human acid alpha-glucosidase isoform 1-M (GAA1-M) (SEQ ID NO:13).

FIG. 6 describes the amino sequence of human acid alpha-glucosidase isoform 1-L (GAA1-L) (SEQ ID NO:14).

FIG. 7 describes the amino sequence of human acid alpha-glucosidase isoform 4 (GAA4) (SEQ ID NO:15).

FIG. 8 describes a method for purifying highly phosphorylated recombinant human acid alpha-glucosidase (rhGAA) from a G71 transfectant (CIN11) grown on microcarrier beads following a three-column purification procedure.

FIG. 9 describes a method for purifying highly phosphorylated recombinant human acid alpha-glucosidase (rhGAA) from a G71 transfectant (1B8) grown in suspension following a three-column purification procedure.

FIG. 10 describes a method for purifying highly phosphorylated recombinant human acid alpha-glucosidase (rhGAA) from a G71 transfectant (F5) grown on microcarrier beads following a four-column purification procedure.

FIG. 11 describes a method for purifying highly phosphorylated recombinant human acid alpha-glucosidase (rhGAA) from a G71 transfectant (C3) grown in suspension following a four-column purification procedure.

FIG. 12 shows FACE analysis of recombinant human acid alpha-glucosidase (rhGAA) expressed by from G715 (G71) and 3.1.36 (DUXB11) cells.

FIG. 13 demonstrates binding of G71 produced recombinant human acid alpha-glucosidase (rhGAA) to a mannose 6-phosphate receptor column.

FIG. 14 compares the uptake of G71 recombinant human acid alpha-glucosidase (rhGAA) and DUX rhGAA into GM244 Pompe fibroblasts.

FIGS. 15A and 15B demonstrate glycogen clearance in the heart and diaphragm, respectively, in a mouse model of Pompe disease upon administration of G71 produced recombinant human acid alpha-glucosidase (rhGAA).

### DETAILED DESCRIPTION OF THE INVENTION

The present invention relates to the discovery of a method that reconciles the need for large-scale manufacture of lysosomal enzymes with the requirement of a highly phosphorylated lysosomal enzyme product that is efficient in targeting lysosomes and hence is therapeutically effective.

In addition to the presence of the mannose 6-phosphate marker on lysosomal enzyme oligosaccharide, lysosomal routing of enzymes depends on the acidification of trafficking endosomes emerging from the end of the trans Golgi stack. Chemical quenching of the acidic environment within these endosomes with diffusible basic molecules results in disgorgement of the vesicular contents, including lysosomal enzymes, into the extracellular milieu (Braulke et al., Eur. J. Cell Biol. 43(3):316-321, 1987). Acidification requires a specific vacuolar ATPase embedded within the membrane of the endosome (Nishi et al., Nat. Rev. Mol. Cell Biol. 3(2):94-103, 2002). Failure of this ATPase is expected to enhance the secretion of lysosomal enzymes at the expense of lysosomal routing. Manufacturing cell lines which carry defects in the vacuolar ATPase would be expected to prevent non-productive diversion of phosphorylated recombinant enzyme to the intracellular lysosomal compartment.

In 1984, Chinese hamster ovary (CHO) cell mutants specifically defective in endosomal acidification were generated and characterized (Park et al., Somat. Cell Mol. Genet. 17(2):137-150, 1991). CHO-K1 cells were chemically mutagenized and selected for survival at elevated temperatures in the presence of toxins. These toxins required endosomal acidification for the full expression of their lethality (Marnell et al., (1984), supra). In the former study, a cocktail of two toxins with different mechanisms of action was chosen to avoid selection of toxin-specific resistance. The principle is that while the probability of serendipitous mutations that result in resistance to one particular toxin is small, the probability of two simultaneous serendipitous mutations specific for two entirely different toxins is non-existent. Selections were carried out at elevated temperature to allow for temperature-sensitive mutations. This genetic screen resulted in two mutants, one of which was designated G.7.1 (G71), that were resistant to toxins at elevated temperatures. The lesion in G71 was not due to the uptake or mechanism of action of the two toxins, but resulted from an inability of the clone to acidify endosomes at elevated temperatures. This inability was also evident at permissive temperatures (34° C.), although to a lesser extent. G71 cells were also found to be auxotrophic for iron at elevated temperatures, despite normal uptake of transferrin from the medium (Timchak et al., J. Biol. Chem. 261(30):14154-14159, 1986). Since iron was released from transferrin only at low pH, auxotrophy for iron despite normal transferrin uptake indicated a failure in endosomal acidification. Another study demonstrated that the acidification defect was manifested primarily in endosomes rather than lysosomes (Stone et al., J. Biol. Chem. 262(20):9883-9886, 1987). The data on G71 were consistent with the conclusion that a mutation resulted in the destabilization of the vacuolar ATPase responsible for endosomal acidification. Destabilization was most evident at elevated temperatures (39.5° C.), but was partially expressed even at lower temperatures (34° C.). A study of the trafficking of two endogenous lysosomal enzymes, cathepsin D and acid alpha-glucosidase, in G71 cells (Park et al., (1991), supra) showed that both enzymes were quantitatively secreted at elevated temperatures, and glycosylation of the enzymes was unaffected. The secretion of phosphorylated acid alpha-glucosidase was significantly enhanced at non-permissive temperatures.

Thus, the ability of G71 cells, mutant CHO cells that are defective in endosomal acidification, to overexpress a human lysosomal enzyme could provide a mechanism for the large-scale production of highly phosphorylated recombinant human lysosomal enzymes.

### I. DEFINITIONS

Unless otherwise defined, all technical and scientific terms used herein have the same meaning as commonly understood by one of ordinary skill in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

As used herein, the following terms have the meanings ascribed to them unless specified otherwise.

“Allelic variant” refers to any of two or more polymorphic forms of a gene occupying the same genetic locus. Allelic variations arise naturally through mutation, and may result in phenotypic polymorphism within populations. Gene mutations can be silent (no change in the encoded polypeptide) or may encode polypeptides having altered amino acid sequences. “Allelic variants” also refer to cDNAs derived from mRNA transcripts of genetic allelic variants, as well as the proteins encoded by them.

“Amplification” refers to any means by which a polynucleotide sequence is copied and thus expanded into a larger number of polynucleotide molecules, e.g., by reverse transcription, polymerase chain reaction, and ligase chain reaction.

A first sequence is an “antisense sequence” with respect to a second sequence if a polynucleotide whose sequence is the first sequence specifically hybridizes with a polynucleotide whose sequence is the second sequence.

“cDNA” refers to a DNA that is complementary or identical to an mRNA, in either single stranded or double stranded form.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′. A nucleotide sequence is “substantially complementary” to a reference nucleotide sequence if the sequence complementary to the subject nucleotide sequence is substantially identical to the reference nucleotide sequence.

“Conservative substitution” refers to the substitution in a polypeptide of an amino acid with a functionally similar amino acid. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (1), Leucine (L), Methionine (M), Valine (V); and
6) Phenylalanine (F), Tyrosine (Y), Tryptophan (W).

The term “derivative” when used in reference to polypeptides refers to polypeptides chemically modified by techniques such as, for example, ubiquitination, labeling (e.g., with radionuclides or various enzymes), covalent polymer attachment such as pegylation (derivatization with polyethylene glycol) and insertion or substitution by chemical synthesis of amino acids such as ornithine, which do not normally occur in human proteins.

The term “derivative” when used in reference to cell lines refers to cell lines that are descendants of the parent cell line; for example, this term includes cells that have been passaged or subcloned from parent cells and retain the desired property, descendants of the parent cell line that have been mutated and selected for retention of the desired property, and descendants of the parent cell line which have been altered to contain different expression vectors or different exogenously added nucleic acids.

“Detecting” refers to determining the presence, absence, or amount of an analyte in a sample, and can include quantifying the amount of the analyte in a sample or per cell in a sample.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantitate the amount of bound detectable moiety in a sample. The detectable moiety can be incorporated in or attached to a primer or probe either covalently, or through ionic, van der Waals or hydrogen bonds, e.g., incorporation of radioactive nucleotides, or biotinylated nucleotides that are recognized by streptavadin. The detectable moiety may be directly or indirectly detectable. Indirect detection can involve the binding of a second directly or indirectly detectable moiety to the detectable moiety. For example, the detectable moiety can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules. (See, e.g., Fahrlander et al., Bio/Technology 6:1165, 1988). Quantitation of the signal is achieved by, e.g., scintillation counting, densitometry, or flow cytometry.

“Diagnostic” means identifying the presence or nature of a pathologic condition. Diagnostic methods differ in their specificity and selectivity. While a particular diagnostic method may not provide a definitive diagnosis of a condition, it suffices if the method provides a positive indication that aids in diagnosis.

The term “effective amount” means a dosage sufficient to produce a desired result on a health condition, pathology, and disease of a subject or for a diagnostic purpose. The desired result may comprise a subjective or objective improvement in the recipient of the dosage. “Therapeutically effective amount” refers to that amount of an agent effective to produce the intended beneficial effect on health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Equivalent dose” refers to a dose, which contains the same amount of active agent.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in vitro expression system. Expression vectors include all those known in the art, such as cosmids, plasmids (e.g., naked or contained in liposomes) and viruses that incorporate the recombinant polynucleotide.

“Highly phosphorylated”, “high level of phosphorylation” and “high level of phosphorylated oligosaccharides” refers to preparations of protein in which at least 70% of the protein binds to the cation-independent mannose 6-phosphate receptor through phosphorylated oligosaccharides. Binding is further characterized by sensitivity to competition with mannose 6-phosphate. A highly phosphorylated enzyme may also refer to an enzyme with at least 0.7 bis-phosphorylated oligomannose chains per mole of protein.

The terms “identical” or percent “identity,” in the context of two or more polynucleotide or polypeptide sequences, refer to two or more sequences or subsequences that are the same or have a specified percentage of nucleotides or amino acid residues that are the same, when compared and aligned for maximum correspondence, as measured using one of the following sequence comparison algorithms or by visual inspection.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

“Low level of phosphorylation” or “low phosphorylation” refers to a preparation of protein in which the uptake into fibroblast cells has a half maximal concentration of greater than 10 nM or the fraction of enzyme that binds a man 6-P receptor column is less than 30-50%.

“Low level of unphosphorylated high-mannose oligosaccharide” refers to a preparation of protein in which each molecule of protein has at least one molecule of complex oligosaccharide in place of a high-mannose oligosaccharide. Complex oligosaccharide contains galactose, acetylglucsamine (GlcNAc) and sialic acid, in addition to other sugars.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man in the laboratory is naturally-occurring.

“Pharmaceutical composition” refers to a composition suitable for pharmaceutical use in subject animal, including humans and mammals. A pharmaceutical composition comprises a pharmacologically effective amount of a therapeutic lysosomal enzyme and also comprises a pharmaceutically acceptable carrier. A pharmaceutical composition encompasses a composition comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a therapeutic lysosomal enzyme of the present invention and a pharmaceutically acceptable carrier.

“Pharmaceutically acceptable carrier” refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration). A “pharmaceutically acceptable salt” is a salt that can be formulated into a compound for pharmaceutical use including, e.g., metal salts (sodium, potassium, magnesium, calcium, etc.) and salts of ammonia or organic amines.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

A “prophylactic” treatment is a treatment administered to a subject who does not exhibit signs of a disease or exhibits only early signs for the purpose of decreasing the risk of developing pathology. The lysosomal enzymes of the invention may be given as a prophylactic treatment to reduce the likelihood of developing a pathology or to minimize the severity of the pathology, if developed.

“Recombinant polynucleotide” refers to a polynucleotide having sequences that are not naturally joined together. An amplified or assembled recombinant polynucleotide may be included in a suitable vector, and the vector can be used to transform a suitable host cell. A host cell that comprises the recombinant polynucleotide is referred to as a “recombinant host cell.” The gene is then expressed in the recombinant host cell to produce, e.g., a “recombinant polypeptide.” A recombinant polynucleotide may serve a non-coding function (e.g., promoter, origin of replication, ribosome-binding site, etc.) as well.

Hybridizing specifically to” or “specific hybridization” or “selectively hybridize to”, refers to the binding, duplexing, or hybridizing of a nucleic acid molecule preferentially to a particular nucleotide sequence under stringent conditions when that sequence is present in a complex mixture (e.g., total cellular) DNA or RNA.

The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes part I chapter 2 “Overview of principles of hybridization and the strategy of nucleic acid probe assays”, Elsevier, New York. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook, et al. for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2× (or higher) than that observed for an unrelated probe in the particular hybridization assay indicates detection of a specific hybridization.

A “subject” of diagnosis or treatment is a human or non-human animal, including a mammal or a primate.

The phrase “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of either or both comparison biopolymers.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl Math. 2:482, 1981, by the homology alignment algorithm of Needleman & Wunsch, J. Mol. Biol. 48:443, 1970, by the search for similarity method of Pearson & Lipman, Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel, et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps. Another algorithm that is useful for generating multiple alignments of sequences is Clustal W (Thompson, et al. CLUSTAL W: improving the sensitivity of progressive multiple sequence alignment through sequence weighting, positions-specific gap penalties and weight matrix choice, Nucleic Acids Research 22: 4673-4680, 1994).

Another example of algorithm that is suitable for determining percent sequence identity and sequence similarity is the BLAST algorithm, which is described in Altschul et al., J. Mol. Biol. 215:403-410, 1990. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, (1990), supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915, 1989).

In addition to calculating percent sequence identity, the BLAST algorithm also performs a statistical analysis of the similarity between two sequences (see, e.g., Karlin & Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described herein.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition. In some embodiments, the conjugates of the invention are substantially pure or isolated. In some embodiments, the conjugates of the invention are substantially pure or isolated with respect to the macromolecular starting materials used in their synthesis. In some embodiments, the pharmaceutical composition of the invention comprises a substantially purified or isolated therapeutic enzyme admixed with one or more pharmaceutically acceptable excipient.

A “therapeutic” treatment is a treatment administered to a subject who exhibits signs or symptoms of pathology for the purpose of diminishing or eliminating those signs or symptoms. The signs or symptoms may be biochemical, cellular, histological, functional, subjective or objective. The lysosmal enzymes of the invention may be given as a therapeutic treatment or for diagnosis.

“Therapeutic index” refers to the dose range (amount and/or timing) above the minimum therapeutic amount and below an unacceptably toxic amount.

“Treatment” refers to prophylactic treatment or therapeutic treatment or diagnostic treatment.

The term “unit dosage form,” as used herein, refers to physically discrete units suitable as unitary dosages for human and animal subjects, each unit containing a predetermined quantity of lysosomal enzymes of the present invention calculated in an amount sufficient to produce the desired effect in association with a pharmaceutically acceptable diluent, carrier or vehicle. The specifications for the novel unit dosage forms of the present invention depend on the particular lysosomal enzyme employed and the effect to be achieved, and the pharmacodynamics associated with each lysosomal enzyme in the host.

### II. PRODUCTION OF LYSOSOMAL ENZYMES

In one aspect, the present invention features a novel method of producing lysosomal enzymes in amounts that enable therapeutic use of such enzymes. In general, the method features transformation of a suitable cell line with the cDNA encoding for full-length lysosomal enzymes or a biologically active fragment, variant, or mutant thereof. Those of skill in the art may prepare expression constructs other than those expressly described herein for optimal production of such lysosomal enzymes in suitable transfected cell lines therewith. Moreover, skilled artisans may easily design fragments of cDNA encoding biologically active fragments, variants, or mutants of the naturally occurring lysosomal enzymes that possess the same or similar biological activity to the naturally occurring full-length enzyme.

### Host Cells

Host cells used to produce proteins are endosomal acidification-deficient cell lines characterized by their ability to produce lysosomal enzymes in amounts that enable use of the enzyme therapeutically. The invention provides a CHO-K1-derived, END3 complementation group cell line, designated G71. The invention also provides G71 cell lines which have been subcloned further or which contain different expression plasmids, designated G715 and G71 GAA2, respectively. The invention also provides G71 cell lines which contain different expression plasmids, designated 1B8, C3, D7 and F5. The invention also provides G71 cell lines and derivatives thereof which have been adapted for growth in suspension, designated G71-S and derivatives thereof.

Cells that contain and express DNA or RNA encoding the chimeric protein are referred to herein as genetically modified cells. Mammalian cells that contain and express DNA or RNA encoding the chimeric protein are referred to as genetically modified mammalian cells. Introduction of the DNA or RNA into cells is by a known transfection method, such as electroporation, microinjection, microprojectile bombardment, calcium phosphate precipitation, modified calcium phosphate precipitation, cationic lipid treatment, photoporation, fusion methodologies, receptor mediated transfer, or polybrene precipitation. Alternatively, the DNA or RNA can be introduced by infection with a viral vector. Methods of production for cells, including mammalian cells, which express DNA or RNA encoding a chimeric protein are described in co-pending patent applications U.S. Ser. No. 08/334,797, entitled “In Vivo Protein Production and Delivery System for Gene Therapy”, by Richard F Selden, Douglas A. Treco and Michael W. Heartlein (filed Nov. 4, 1994); U.S. Ser. No. 08/334,455, entitled “In Vivo Production and Delivery of Erythropoietin or Insulinotropin for Gene Therapy”, by Richard F Selden, Douglas A. Treco and Michael W. Heartlein (filed Nov. 4, 1994) and U.S. Ser. No. 08/231,439, entitled “Targeted Introduction of DNA Into Primary or Secondary Cells and Their Use for Gene Therapy”, by Douglas A. Treco, Michael W. Heartlein and Richard F Selden (filed Apr. 20, 1994). The teachings of each of these applications are expressly incorporated herein by reference in their entirety.

In preferred embodiments, the host cell used to produce proteins is an endosomal acidification-deficient cell line characterized by its ability to produce lysosomal enzymes in amounts that enable use of the enzyme therapeutically. In preferred embodiments, the invention provides a CHO-K1-derived, END3 complementation group cell line, designated G71, that is capable of producing high yields of highly phosphorylated lysosomal enzymes, as specified in “DEFINITIONS”, thereby enabling the large scale production of therapeutic lysosomal enzymes. In most preferred embodiments, the cell line expresses and secretes recombinant lysosomal enzymes in amounts of at least about 0.25 picogram/cell/day, preferably at least about 0.5 picogram/cell/day, more preferably at least about 1.0 picogram/cell/day, or more.

### Vectors and Nucleic Acid Constructs

A nucleic acid construct used to express the chimeric protein can be one which is expressed extrachromosomally (episomally) in the transfected mammalian cell or one which integrates, either randomly or at a pre-selected targeted site through homologous recombination, into the recipient cell's genome. A construct which is expressed extrachromosomally comprises, in addition to chimeric protein-encoding sequences, sequences sufficient for expression of the protein in the cells and, optionally, for replication of the construct. It typically includes a promoter, chimeric protein-encoding DNA and a polyadenylation site. The DNA encoding the chimeric protein is positioned in the construct in such a manner that its expression is under the control of the promoter. Optionally, the construct may contain additional components such as one or more of the following: a splice site, an enhancer sequence, a selectable marker gene under the control of an appropriate promoter, and an amplifiable marker gene under the control of an appropriate promoter.

In those embodiments in which the DNA construct integrates into the cell's genome, it need include only the chimeric protein-encoding nucleic acid sequences. Optionally, it can include a promoter and an enhancer sequence, a polyadenylation site or sites, a splice site or sites, nucleic acid sequences which encode a selectable marker or markers, nucleic acid sequences which encode an amplifiable marker and/or DNA homologous to genomic DNA in the recipient cell, to target integration of the DNA to a selected site in the genome (to target DNA or DNA sequences).

### Cell Culture Methods

Mammalian cells containing the chimeric protein-encoding DNA or RNA are cultured under conditions appropriate for growth of the cells and expression of the DNA or RNA. Those cells which express the chimeric protein can be identified, using known methods and methods described herein, and the chimeric protein can be isolated and purified, using known methods and methods also described herein, either with or without amplification of chimeric protein production. Identification can be carried out, for example, through screening genetically modified mammalian cells that display a phenotype indicative of the presence of DNA or RNA encoding the chimeric protein, such as PCR screening, screening by Southern blot analysis, or screening for the expression of the chimeric protein. Selection of cells which contain incorporated chimeric protein-encoding DNA may be accomplished by including a selectable marker in the DNA construct, with subsequent culturing of transfected or infected cells containing a selectable marker gene, under conditions appropriate for survival of only those cells that express the selectable marker gene. Further amplification of the introduced DNA construct can be affected by culturing genetically modified mammalian cells under appropriate conditions (e.g., culturing genetically modified mammalian cells containing an amplifiable marker gene in the presence of a concentration of a drug at which only cells containing multiple copies of the amplifiable marker gene can survive).

Genetically modified mammalian cells expressing the chimeric protein can be identified, as described herein, by detection of the expression product. For example, mammalian cells expressing highly phosphorylated enzymes can be identified by a sandwich enzyme immunoassay. The antibodies can be directed toward the active agent portion.

### Variants of Lysosomal Enzymes

In certain embodiments, highly phosphorylated lysosomal enzyme analogs and variants may be prepared and will be useful in a variety of applications in which highly phosphorylated lysosomal enzymes may be used. Amino acid sequence variants of the polypeptide can be substitutional, insertional or deletion variants. Deletion variants lack one or more residues of the native protein which are not essential for function or immunogenic activity. A common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, also called fusion proteins, are discussed below.

Variants may be substantially homologous or substantially identical to the unmodified lysosomal enzyme as set out above. Preferred variants are those which are variants of a highly phosphorylated lysosomal enzyme polypeptide which retain at least some of the biological activity, e.g. catalytic activity, of the lysosomal enzyme. Other preferred variants include variants of a polypeptide of acid alpha-glucosidase which retain at least about 50%, preferably at least about 75%, more preferably at least about 90%, of the catalytic activity of the acid alpha-glucosidase.

Substitutional variants typically exchange one amino acid of the wild-type for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

One aspect of the present invention contemplates generating glycosylation site mutants in which the O- or N-linked glycosylation site of the lysosomal enzyme protein has been mutated. Such mutants will yield important information pertaining to the biological activity, physical structure and substrate binding potential of the highly phosphorylated lysosomal enzyme. In particular aspects it is contemplated that other mutants of the highly phosphorylated lysosomal enzyme polypeptide may be generated that retain the biological activity but have increased or decreased substrate binding activity. As such, mutations of the active site or catalytic region are particularly contemplated in order to generate protein variants with altered substrate binding activity. In such embodiments, the sequence of the highly phosphorylated lysosomal enzyme is compared to that of the other related enzymes and selected residues are specifically mutated.

Numbering the amino acids of the mature protein (e.g., recombinant human acid alpha-glucosidase or rhGAA) from the putative amino terminus as amino acid number 1, exemplary mutations that may be useful include, for example, deletion of all or some of glycosylated asparagines, including N140, N233, N390, N470, N652, N882 and N925 (Hermans et al., Biochem. J. 289 (Pt 3):681-686, 1993). Substrate binding can be modified by mutations at D91 (the amino acid that differs between alleles GAA1 and GAA2 (Swallow et al., Ann. Hum. Genet. 53 (Pt 2):177-178, 1989). Taking into consideration such mutations are exemplary, those of skill in the art will recognize that other mutations of the enzyme sequence can be made to provide additional structural and functional information about this protein and its activity.

In order to construct mutants such as those described above, one of skill in the art may employ well known standard technologies. Specifically contemplated are N-terminal deletions, C-terminal deletions, internal deletions, as well as random and point mutagenesis.

N-terminal and C-terminal deletions are forms of deletion mutagenesis that take advantage for example, of the presence of a suitable single restriction site near the end of the C- or N-terminal region. The DNA is cleaved at the site and the cut ends are degraded by nucleases such as BAL31, exonuclease III, DNase I, and S1 nuclease. Rejoining the two ends produces a series of DNAs with deletions of varying size around the restriction site. Proteins expressed from such mutant can be assayed for appropriate biological function, e.g. enzymatic activity, using techniques standard in the art, and described in the specification. Similar techniques may be employed for internal deletion mutants by using two suitably placed restriction sites, thereby allowing a precisely defined deletion to be made, and the ends to be religated as above.

Also contemplated are partial digestion mutants. In such instances, one of skill in the art would employ a “frequent cutter” that cuts the DNA in numerous places depending on the length of reaction time. Thus, by varying the reaction conditions it will be possible to generate a series of mutants of varying size, which may then be screened for activity.

A random insertional mutation may also be performed by cutting the DNA sequence with a DNase I, for example, and inserting a stretch of nucleotides that encode, 3, 6, 9, 12 etc., amino acids and religating the end. Once such a mutation is made the mutants can be screened for various activities presented by the wild-type protein.

Point mutagenesis also may be employed to identify with particularity which amino acid residues are important in particular activities associated with lysosomal enzyme biological activity. Thus, one of skill in the art will be able to generate single base changes in the DNA strand to result in an altered codon and a missense mutation.

The amino acids of a particular protein can be altered to create an equivalent, or even an improved, second-generation molecule. Such alterations contemplate substitution of a given amino acid of the protein without appreciable loss of interactive binding capacity with structures such as, for example, antigen-binding regions of antibodies or binding sites on substrate molecules or receptors. Since it is the interactive capacity and nature of a protein that defines that protein's biological functional activity, certain amino acid substitutions can be made in a protein sequence, and its underlying DNA coding sequence, and nevertheless obtain a protein with like properties. Thus, various changes can be made in the DNA sequences of genes without appreciable loss of their biological utility or activity, as discussed below.

In making such changes, the hydropathic index of amino acids may be considered. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics (Kyte & Doolittle, J. Mol. Biol. 157(1):105-132, 1982, incorporated herein by reference). Generally, amino acids may be substituted by other amino acids that have a similar hydropathic index or score and still result in a protein with similar biological activity, i.e., still obtain a biological functionally equivalent protein.

In addition, the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As such, an amino acid can be substituted for another having a similar hydrophilicity value and still obtain a biologically equivalent and immunologically equivalent protein.

Exemplary amino acid substitutions that may be used in this context of the invention include but are not limited to exchanging arginine and lysine; glutamate and aspartate; serine and threonine; glutamine and asparagine; and valine, leucine and isoleucine. Other such substitutions that take into account the need for retention of some or all of the biological activity whilst altering the secondary structure of the protein will be well known to those of skill in the art.

Another type of variant that is contemplated for the preparation of polypeptides according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles described above, to engineer second generation molecules having many of the natural properties of lysosomal enzymes, but with altered and even improved characteristics.

### Modified Glycosylation

Variants of a highly phosphorylated lysosomal enzyme can also be produced that have a modified glycosylation pattern relative to the parent polypeptide, for example, deleting one or more carbohydrate moieties, and/or adding one or more glycosylation sites that are not present in the native polypeptide.

Glycosylation is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. The presence of either of these tripeptide sequences in a polypeptide creates a potential glycosylation site. Thus, N-linked glycosylation sites may be added to a polypeptide by altering the amino acid sequence such that it contains one or more of these tripeptide sequences. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. O-linked glycosylation sites may be added by inserting or substituting one or more serine or threonine residues to the sequence of the original polypeptide.

### Domain Switching

Various portions of lysosomal enzyme proteins possess a great deal of sequence homology. Mutations may be identified in lysosomal enzyme polypeptides which may alter its function. These studies are potentially important for at least two reasons. First, they provide a reasonable expectation that still other homologs, allelic variants and mutants of this gene may exist in related species, such as rat, rabbit, monkey, gibbon, chimp, ape, baboon, cow, pig, horse, sheep and cat. Upon isolation of these homologs, variants and mutants, and in conjunction with other analyses, certain active or functional domains can be identified. Second, this will provide a starting point for further mutational analysis of the molecule as described above. One way in which this information can be exploited is in “domain switching.”

Domain switching involves the generation of chimeric molecules using different but related polypeptides. For example, by comparing the sequence of a lysosomal enzyme, e.g. acid alpha-glucosidase, with that of a similar lysosomal enzyme from another source and with mutants and allelic variants of these polypeptides, one can make predictions as to the functionally significant regions of these molecules. It is possible, then, to switch related domains of these molecules in an effort to determine the criticality of these regions to enzyme function and effects in lysosomal storage disorders. These molecules may have additional value in that these “chimeras” can be distinguished from natural molecules, while possibly providing the same or even enhanced function.

Based on the numerous lysosomal enzymes now being identified, further analysis of mutations and their predicted effect on secondary structure will add to this understanding. It is contemplated that the mutants that switch domains between the lysosomal enzymes will provide useful information about the structure/function relationships of these molecules and the polypeptides with which they interact.

### Fusion Proteins

In addition to the mutations described above, the present invention further contemplates the generation of a specialized kind of insertional variant known as a fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

There are various commercially available fusion protein expression systems that may be used in the present invention. Particularly useful systems include but are not limited to the glutathione S-transferase (GST) system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.). These systems are capable of producing recombinant polypeptides bearing only a small number of additional amino acids, which are unlikely to affect the antigenic ability of the recombinant polypeptide. For example, both the FLAG system and the 6×His system add only short sequences, both of which are known to be poorly antigenic and which do not adversely affect folding of the polypeptide to its native conformation. Another N-terminal fusion that is contemplated to be useful is the fusion of a Met-Lys dipeptide at the N-terminal region of the protein or peptides. Such a fusion may produce beneficial increases in protein expression or activity.

A particularly useful fusion construct may be one in which a highly phosphorylated lysosomal enzyme polypeptide or fragment thereof is fused to a hapten to enhance immunogenicity of a lysosomal enzyme fusion construct. This may be useful in the production of antibodies to the highly phosphorylated lysosomal enzyme to enable detection of the protein. In other embodiments, fusion construct can be made which will enhance the targeting of the lysosomal enzyme-related compositions to a specific site or cell.

Other fusion constructs including a heterologous polypeptide with desired properties, e.g., an Ig constant region to prolong serum half life or an antibody or fragment thereof for targeting also are contemplated. Other fusion systems produce polypeptide hybrids where it is desirable to excise the fusion partner from the desired polypeptide. In one embodiment, the fusion partner is linked to the recombinant highly phosphorylated lysosomal enzyme polypeptide by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

### Derivatives

As stated above, derivative refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as ornithine. Derivatives of the lysosomal enzyme are also useful as therapeutic agents and may be produced by the method of the invention.

Polyethylene glycol (PEG) may be attached to the lysosomal enzyme produced by the method of the invention to provide a longer half-life in vivo. The PEG group may be of any convenient molecular weight and may be linear or branched. The average molecular weight of the PEG will preferably range from about 2 kiloDaltons (“kDa”) to about 100 kDa, more preferably from about 5 kDa to about 50 kDa, most preferably from about 5 kDa to about 10 kDa. The PEG groups will generally be attached to the lysosomal enzymes of the invention via acylation or reductive alkylation through a reactive group on the PEG moiety (e.g., an aldehyde, amino, thiol, or ester group) to a reactive group on the inventive lysosomal enzyme (e.g., an aldehyde, amino, or ester group). Addition of PEG moieties to polypeptide of interest can be carried out using techniques well-known in the art. See, e.g., International Publication No. WO 96/11953 and U.S. Pat. No. 4,179,337.

Ligation of the enzyme polypeptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

### Labels

In some embodiments, the therapeutic enzyme is labeled to facilitate its detection. A “label” or a “detectable moiety” is a composition detectable by spectroscopic, photochemical, biochemical, immunochemical, chemical, or other physical means. For example, labels suitable for use in the present invention include, radioactive labels (e.g., ³²P), fluorophores (e.g., fluorescein), electron-dense reagents, enzymes (e.g., as commonly used in an ELISA), biotin, digoxigenin, or haptens as well as proteins which can be made detectable, e.g., by incorporating a radiolabel into the hapten or peptide, or used to detect antibodies specifically reactive with the hapten or peptide.

Examples of labels suitable for use in the present invention include, but are not limited to, fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold, colored glass or plastic beads (e.g., polystyrene, polypropylene, latex, etc.).

The label may be coupled directly or indirectly to the desired component of the assay according to methods well known in the art. Preferably, the label in one embodiment is covalently bound to the biopolymer using an isocyanate reagent for conjugation of an active agent according to the invention. In one aspect of the invention, the bifunctional isocyanate reagents of the invention can be used to conjugate a label to a biopolymer to form a label biopolymer conjugate without an active agent attached thereto. The label biopolymer conjugate may be used as an intermediate for the synthesis of a labeled conjugate according to the invention or may be used to detect the biopolymer conjugate. As indicated above, a wide variety of labels can be used, with the choice of label depending on sensitivity required, ease of conjugation with the desired component of the assay, stability requirements, available instrumentation, and disposal provisions. Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound.

The lysosomal enzymes of the invention can also be conjugated directly to signal-generating compounds, e.g., by conjugation with an enzyme or fluorophore. Enzymes suitable for use as labels include, but are not limited to, hydrolases, particularly phosphatases, esterases and glycosidases, or oxidotases, particularly peroxidases. Fluorescent compounds, i.e., fluorophores, suitable for use as labels include, but are not limited to, fluorescein and its derivatives, rhodamine and its derivatives, dansyl, umbelliferone, etc. Further examples of suitable fluorophores include, but are not limited to, eosin, TRITC-amine, quinine, fluorescein W, acridine yellow, lissamine rhodamine, B sulfonyl chloride erythroscein, ruthenium (tris, bipyridinium), Texas Red, nicotinamide adenine dinucleotide, flavin adenine dinucleotide, etc. Chemiluminescent compounds suitable for use as labels include, but are not limited to, luciferin and 2,3-dihydrophthalazinediones, e.g., luminol. For a review of various labeling or signal producing systems that can be used in the methods of the present invention, see U.S. Pat. No. 4,391,904.

Means for detecting labels are well known to those of skill in the art. Thus, for example, where the label is radioactive, means for detection include a scintillation counter or photographic film, as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Other labeling and detection systems suitable for use in the methods of the present invention will be readily apparent to those of skill in the art. Such labeled modulators and ligands can be used in the diagnosis of a disease or health condition.

In a preferred embodiment, the method comprises the step of producing highly phosphorylated lysosomal enzymes from cell lines with defects in endosomal trafficking. In a particularly preferred embodiment, the method comprises the step of producing highly phosphorylated recombinant human acid alpha-glucosidase (rhGAA) from the CHO cell line, G71. Production of lysosomal enzymes comprises the steps of: (a) development of recombinant G71 expressing acid alpha-glucosidase (GAA); (b) culture of the cells; and (c) scaling up of cell line to bioreactor for production of lysosomal enzymes. In preferred embodiments, human GAA is amplified from human liver mRNA (Clontech 6510-1) and subcloned into the mammalian expression vector pCINt (BioMarin). The vector pCINt comprises the human CMV enhancer-promoter, rabbit β-globin IVS2 intron, multiple cloning site from pcDNA3.1 (+) (Invitrogen), bovine growth hormone poly-adenylation signal for efficient transcript termination, and selection marker neomycin phosphotransferase gene with a point mutation to decrease enzyme efficiency. The attenuated marker is further handicapped with the weak HSV-tk promoter. In another preferred embodiment, the human GAA cDNA is amplified and subcloned into the mammalian expression vector pBMP14 EIN (BioMarin). pBMP14 EIN is similar to vector pCINt, except that the human elongation factor-1α promoter replaces the human CMV enhancer-promoter.

For cell line development, in a first series of transfections, G71 was transfected with linearized expression plasmids derived from pCINt (BioMarin) and recombinants were selected. After a first round of subcloning of transfectants, four cell lines were selected using the fluorescent substrate and specifically designated. CIN cell lines were analyzed for cell-specific productivity (pg of product/cell) in spinners with microcarriers. Cell lines were cultured in JRH Excell 302 medium supplemented with 2 mM glutamine and 5% fetal calf serum, seeded onto Cytopore microcarriers and grown in 200 mL spinner flasks. Serum was removed by dilution over the course of a week until BSA was undetectable by ELISA. The best producer was identified and scaled-up to bioreactor for production of pre-clinical material.

In a second series of transfections, G71 was transfected with linearized expression plasmids derived from pBMP 14 EIN and recombinants were selected. rhGAA producing G71 cell lines were selected, analyzed, cultured and scaled-up for production as described supra. G71 transfectants were also adapted to suspension as described infra.

### III. PURIFICATION OF LYSOSOMAL ENZYMES

The invention provides methods to purify lysosomal enzymes, e.g., recombinant human acid alpha-glucosidase (rhGAA), produced by the methods described herein. In one embodiment, the invention provides a method for purifying a lysosomal enzyme comprising the steps of: (a) dia-filtering a cell harvest medium containing the lysosomal enzyme, (b) adjusting the pH of the dia-filtered harvest to 5.5 and then to 4.5 to induce precipitation of contaminating proteins, (c) re-filtering the pH adjusted dia-filtered harvest to remove precipitate; and (d) isolating the lysosomal enzyme by sequential chromatography comprising the steps of: (i) loading the filtrate from step (c) onto Blue-Sepharose, washing with 20 mM acetate/phosphate, 50 mM NaCl, pH 4.5 and eluting with 20 mM acetate/phosphate, 50 mM NaCl, pH 5.9; (ii) loading the eluate from step (i) onto Q-Sepharose, washing with 10 mM histidine, pH 6.0, 70 mM NaCl and eluting with 10 mM histidine, pH 6.0, 165 mM NaCl; (iii) adjusting the salt concentration and pH of the eluate from step (ii) to 1.3M NaCl and 5.0, respectively; (and iv) loading the salt and pH adjusted eluate from step (iiii) onto Phenyl-Sepharose and gradient eluting with 1.3M to 0.5 M NaCl. In a preferred embodiment, the lysosomal enzyme is recombinant human acid alpha-glucosidase (rhGAA).

In another embodiment, the invention provides a method for purifying a lysosomal enzyme comprising the steps of: (a) ultrafiltering a cell harvest medium containing the lysosomal enzyme, (b) adjusting the pH of the ultrafiltered harvest to 4.5 to induce precipitation of contaminating proteins; (c) re-filtering the pH adjusted ultrafiltered harvest to remove precipitate; and (d) isolating the lysosomal enzyme by sequential chromatography comprising the steps of: (i) loading the filtrate from step (c) onto Blue-Sepharose, washing with 20 mM acetate/phosphate, 50 mM NaCl, pH 4.5 and eluting with 20 mM acetate/phosphate, 50 mM NaCl, pH 5.9; (ii) loading the eluate from step (i) onto Q-Sepharose, washing with 10 mM histidine, pH 6.0, 70 mM NaCl and eluting with 10 mM histidine, pH 6.0, 165 mM NaCl; (iii) loading the eluate from step (ii) onto SE Hi-CAP, washing with 10 mM acetate, 1 mM phosphate, 5 mM NaCl, pH 5.0 and eluting with a gradient of 5-300 mM NaCl in 10 mM acetate, 1 mM phosphate, pH 5.0, wherein the lysosomal enzyme, i.e., acid-alpha-glucosidase, is present in the flow-through, wash and early part of the salt elution gradient; (iv) adjusting the salt concentration of pooled fractions containing lysosomal enzyme to 2 M NaCl; (v) loading the salt adjusted eluate from step (iv) onto Phenyl-Sepharose and gradient eluting with 1.3M to 0.5 M NaCl; and (vi) formulating the eluate from step (v) in pH 5.8 buffer. In a preferred embodiment, the lysosomal enzyme is recombinant human acid alpha-glucosidase (rhGAA).

The chromatography resins listed above may be substituted with resins belonging to the same or similar class. For example, the Blue-Sepharose resin (i.e., Blue Sepharose 6 FF) may be replaced with other Blue resins, including for example and not for limitation Capto Blue, ToyoPearl Blue, and the like. The Q-Sepharose resin, an anion exchange resin, may be replaced with other anion exchange resins, including for example and not for limitation DEAE-Sepharose, and the like. The SE Hi-CAP resin, a cation exchange resin, may be replaced with other cation exchange resins, including for example and not for limitation Capto S, Giga Cap S, SP-Sepharose, and the like. The Phenyl-Sepharose resin, a hydrophobic interaction chemistry resin, may be replaced with other hydrophobic interaction chemistry resins, including for example and not for limitation ToyoPearl Butyl 650M, Phenyl-Sepharose Hi-Sub, ToyoPearl PPG 600M, Fractogel EMD, and the like. Alternatively, multi-modal resins can replace the anion exchange and/or hydrophobic interaction chemistry resins, including for example and not for limitation Capto MMC, Capto Adhere, PPA Hypercel, and the like.

### IV. LYSOSOMAL ENZYMES AND LYSOSOMAL STORAGE DISEASES

The lysosomal enzyme is a full-length enzyme or any fragment, variant, derivative or mutant thereof that still retains some, substantially all, or all of the therapeutic or biological activity of the enzyme. In some embodiments, the enzyme is one that, if not expressed or produced, or if substantially reduced in expression or production, would give rise to a disease, including but not limited to, lysosomal storage diseases. In some embodiments, the enzyme is one that is not expressed or produced, or is substantially reduced in expression or production, in a lysosomal storage disease (i.e., the reduction or absence of the enzyme is associated with a lysosomal storage disease). Preferably, the enzyme is derived or obtained from a human.

Preferably, the enzyme is a lysosomal storage enzyme, such as alpha-L-iduronidase, iduronate-2-sulfatase, heparan N-sulfatase, alpha-N-acetylglucosaminidase, arylsulfatase A, galactosylceramidase, acid-alpha-glucosidase, thioesterase, hexosaminidase A, acid sphingomyelinase, alpha-galactosidase, or any other lysosomal storage enzyme. Table 1 lists lysosomal storage diseases and the proteins deficient therein, which are useful as a full-length enzyme or any fragment, variant, derivative or mutant thereof that still retains some, substantially all, or all of the therapeutic or biological activity of the enzyme for the treatment of a lysosomal storage disease.

TABLE 1
Proteins Deficient in Lysosomal Storage Diseases

| Lysosomal Storage Disease | Protein Deficiency |
|---|---|
| Mucopolysaccharidosis type I | L-Iduronidase |
| Mucopolysaccharidosis type II Hunter syndrome | Iduronate-2-sulfatase |
| Mucopolysaccharidosis type IIIA Sanfilippo | Heparan-N-sulfatase |
| syndrome | |
| Mucopolysaccharidosis type IIIB Sanfilippo | α-N-Acetylglucosaminidase |
| syndrome | |
| Mucopolysaccharidosis type IIIC Sanfilippo | AcetylCoA:N-acetyltransferase |
| syndrome | |
| Mucopolysaccharidosis type IIID Sanfilippo | N-Acetylglucosamine 6-sulfatase |
| syndrome | |
| Mucopolysaccharidosis type IVA Morquio syndrome | Galactose 6-sulfatase |
| Mucopolysaccharidosis type IVB Morquio syndrome | β-Galactosidase |
| Mucopolysaccharidosis type VI | N-Acetylgalactosamine 4-sulfatase |
| Mucopolysaccharidosis type VII Sly syndrome | β-Glucuronidase |
| Mucopolysaccharidosis type IX | Hyaluronoglucosaminidase |
| Aspartylglucosaminuria | Aspartylglucosaminidase |
| Cholesterol ester storage disease/Wolman disease | Acid lipase |
| Cystinosis | Cystine transporter |
| Danon disease | Lamp-2 |
| Fabry disease | α-Galactosidase A |
| Farber Lipogranulomatosis/Farber disease | Acid ceramidase |
| Fucosidosis | α-L-Fucosidase |
| Galactosialidosis types I/II | Protective protein |
| Gaucher disease types I/IIIII Gaucher disease | Glucocerebrosidase (β-glucosidase) |
| Globoid cell leukodystrophy/Krabbe disease | Galactocerebrosidase |
| Glycogen storage disease II/Pompe disease | α-Glucosidase |
| GM1-Gangliosidosis types I/II/III | β-Galactosidase |
| GM2-Gangliosidosis type I/Tay Sachs disease | β-Hexosaminidase A |
| GM2-Gangliosidosis type II Sandhoff disease | β-Hexosaminidase A |
| GM2-Gangliosidosis | GM2-activator deficiency |
| α-Mannosidosis types I/II | α-D-Mannosidase |
| β-Mannosidosis | β-D-Mannosidase |
| Metachromatic leukodystrophy | Arylsulfatase A |
| Metachromatic leukodystrophy | Saposin B |
| Mucolipidosis type I/Sialidosis types I/II | Neuraminidase |
| Mucolipidosis types II/III I-cell disease | Phosphotransferase |
| Mucolipidosis type IIIC pseudo-Hurler polydystrophy | Phosphotransferase γ-subunit |
| Multiple sulfatase deficiency | Multiple sulfatases |
| Neuronal Ceroid Lipofuscinosis, CLN1 Batten | Palmitoyl protein thioesterase |
| disease | |
| Neuronal Ceroid Lipofuscinosis, CLN2 Batten | Tripeptidyl peptidase I |
| disease | |
| Niemann-Pick disease types A/B Niemann-Pick | Acid sphingomyelinase |
| disease | |
| Niemann-Pick disease type C1 Niemann-Pick disease | Cholesterol trafficking |
| Niemann-Pick disease type C2 Niemann-Pick disease | Cholesterol trafficking |
| Pycnodysostosis | Cathepsin K |
| Schindler disease types I/II Schindler disease | α-Galactosidase B |
| Sialic acid storage disease | Sialic acid transporter |

In preferred embodiments, the enzyme is a recombinant human lysosomal enzyme produced by an endosomal acidification-deficient cell line. In more preferred embodiments, the recombinant human lysosomal enzyme has a high level of phosphorylated oligosaccharides and low level of unphosphorylated high-mannose oligosaccharides as specified under “DEFINITIONS”. In most preferred embodiments, the enzyme is a highly phosphorylated recombinant human acid alpha-glucosidase (rhGAA).

Thus, the lysosomal storage diseases that can be treated or prevented using the methods of the present invention include, but are not limited to, Mucopolysaccharidosis I (MPS I), MPS II, MPS IIIA, MPS IIIB, Metachromatic Leukodystrophy (MLD), Krabbe, Pompe, Neuronal Ceroid Lipofuscinosis CLN2 Batten Disease, Tay-Sachs, Niemann-Pick A and B, and other lysosomal storage diseases.

Thus, per the above table, for each disease the enzyme would preferably comprise a specific lysosomal enzyme deficient or reduced in expression or production in the lysosomal storage disease. For instance, for methods involving MPS I, the enzyme is α-L-iduronidase. For methods involving MPS II, the preferred enzyme is iduronate-2-sulfatase. For methods involving MPS IIIA, the preferred enzyme is heparan N-sulfatase. For methods involving MPS IIIB, the preferred enzyme is α-N-acetylglucosaminidase. For methods involving Metachromatic Leukodystropy (MLD), the preferred enzyme is arylsulfatase A. For methods involving Krabbe, the preferred enzyme is galactosylceramidase. For methods involving Pompe, the preferred enzyme is acid α-glucosidase. For methods involving Neuronal Ceroid Lipofuscinosis CLN2 Batten disease, the preferred enzyme is tripeptidyl peptidase I. For methods involving Tay-Sachs, the preferred enzyme is hexosaminidase alpha. For methods involving Niemann-Pick A and B the preferred enzyme is acid sphingomyelinase.

### V. PHARMACEUTICAL COMPOSITIONS AND ADMINISTRATION

The lysosomal enzymes of the invention may be administered by a variety of routes. For oral preparations, the lysosomal enzymes can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The lysosomal enzymes of the invention can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The lysosomal enzymes of the invention can be utilized in aerosol formulation to be administered via inhalation. The lysosomal enzymes of the present invention can be formulated into pressurized acceptable propellants such as dichlorodifluoromethane, propane, nitrogen and the like.

Furthermore, the lysosomal enzymes of the invention can be made into suppositories by mixing with a variety of bases such as emulsifying bases or water-soluble bases. The lysosomal enzymes of the present invention can be administered rectally via a suppository. The suppository can include vehicles such as cocoa butter, carbowaxes and polyethylene glycols, which melt at body temperature, yet are solidified at room temperature.

Unit dosage forms of the lysosomal enzymes for oral or rectal administration such as syrups, elixirs, and suspensions may be provided wherein each dosage unit, for example, teaspoonful, tablespoonful, tablet or suppository, contains a predetermined amount of the composition containing a lysosomal enzyme. Similarly, unit dosage forms for injection or intravenous administration may comprise the lysosomal enzyme in a composition as a solution in sterile water, normal saline or another pharmaceutically acceptable carrier.

In practical use, the lysosomal enzymes of the invention can be combined as the active ingredient in intimate admixture with a pharmaceutical carrier according to conventional pharmaceutical compounding techniques. The carrier may take a wide variety of forms depending on the preferable form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, for example, suspensions, elixirs and solutions; or carriers such as starches, sugars, microcrystalline cellulose, diluents, granulating agents, lubricants, binders, disintegrating agents and the like in the case of oral solid preparations, for example, powders, hard and soft capsules and tablets, with the solid oral preparations being preferred over the liquid preparations.

With respect to transdermal routes of administration, methods for transdermal administration of drugs are disclosed in Remington's Pharmaceutical Sciences, 17th Edition, (Gennaro et al., Eds., Mack Publishing Co., 1985). Dermal or skin patches are a preferred means for transdermal delivery of the lysosomal enzymes of the invention. Patches preferably provide an absorption enhancer such as DMSO to increase the absorption of the lysosomal enzymes. Other methods for transdermal drug delivery are disclosed in U.S. Pat. Nos. 5,962,012, 6,261,595, and 6,261,595, each of which is incorporated by reference in its entirety.

Pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are also commercially available.

In each of these aspects, the compositions include, but are not limited to, compositions suitable for oral, rectal, topical, parenteral (including subcutaneous, intramuscular, and intravenous), pulmonary (nasal or buccal inhalation), or nasal administration, although the most suitable route in any given case will depend in part on the nature and severity of the conditions being treated and on the nature of the active ingredient. Exemplary routes of administration are the oral and intravenous routes. The compositions may be conveniently presented in unit dosage form and prepared by any of the methods well-known in the art of pharmacy.

In practical use, the lysosomal enzymes according to the invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like; in the case of oral liquid preparations, such as, for example, suspensions, elixirs and solutions; or carriers such as starches, sugars, microcrystalline cellulose, diluents, granulating agents, lubricants, binders, disintegrating agents and the like; in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. The percentage of an active lysosomal enzyme in these compositions may, of course, be varied and may conveniently be between about 2 percent to about 60 percent of the weight of the unit.

The lysosomal enzymes of the invention are useful for therapeutic, prophylactic and diagnostic intervention in animals, and particularly in humans. As described herein, the lysosomal enzymes show preferential accumulation and/or release of the lysosomal enzyme in any target organ, compartment, or site depending upon the lysosomal enzyme used.

Lysosomal enzyme compositions of the present invention may be administered encapsulated in or attached to viral envelopes or vesicles, or incorporated into cells. Vesicles are micellular particles which are usually spherical and which are frequently lipidic. Liposomes are vesicles formed from a bilayer membrane. Suitable vesicles include, but are not limited to, unilamellar vesicles and multilamellar lipid vesicles or liposomes. Such vesicles and liposomes may be made from a wide range of lipid or phospholipid compounds, such as phosphatidylcholine, phosphatidic acid, phosphatidylserine, phosphatidylethanolamine, sphingomyelin, glycolipids, gangliosides, etc. using standard techniques, such as those described in, e.g., U.S. Pat. No. 4,394,448. Such vesicles or liposomes may be used to administer lysosomal enzymes intracellularly and to deliver the lysosomal enzymes to the target organs. Controlled release of a p97-composition of interest may also be achieved using encapsulation (see, e.g., U.S. Pat. No. 5,186,941).

Any route of administration that dilutes the lysosomal enzyme composition into the blood stream, or preferably, at least outside of the blood-brain barrier, may be used. Preferably, the lysosomal enzyme composition is administered peripherally, most preferably intravenously or by cardiac catheter. Intrajugular and intracarotid injections are also useful. Lysosomal enzyme compositions may be administered locally or regionally, such as intraperitoneally, subcutaneously or intramuscularly. In one aspect, lysosomal enzyme compositions are administered with a suitable pharmaceutical diluent or carrier.

Lysosomal enzyme compositions may be administered to bypass the blood-brain barrier, such as, for example and not for limitation, by direct intra-cranial injection, by transient permeabilization of the blood-brain barrier, and by modification of the lysosomal enzyme to alter its tissue distribution. Lysosomal enzyme compositions may be administered by intrathecal injection (i.e., directly into the cerebrospinal fluid). See, e.g., Kakkis, U.S. Patent Application Publication No. 2005/0048047, the disclosure of which is herein incorporated by reference in its entirety.

Those of skill will readily appreciate that dose levels can vary as a function of the specific lysosomal enzyme, the severity of the symptoms and the susceptibility of the subject to side effects. Preferred dosages for a given lysosomal enzyme are readily determinable by those of skill in the art by a variety of means including, but not limited to, dose response and pharmacokinetic assessments conducted in patients, test animals, and in vitro.

Dosages to be administered may also depend on individual needs, on the desired effect, the lysosomal enzyme used and on the chosen route of administration. Preferred dosages of a lysosomal enzyme range from about 0.2 pmol/kg to about 25 nmol/kg, more preferred dosages range from about 2 to 2500 pmol/kg, and particularly preferred dosages range from about 2 to 250 pmol/kg; alternatively, preferred doses of the lysosomal enzyme range from about 0.02 to 2000 mg/kg, more preferred dosages range from about 0.2 to 200 mg/kg, and particularly preferred dosages range from 1 to 40 mg/kg. These dosages will be influenced by the number of lysosomal enzymes associated with the biopolymer. Alternatively, dosages may be calculated based on the lysosomal enzyme administered.

The lysosomal enzymes of the invention are useful for therapeutic, prophylactic and diagnostic intervention in animals, and in particular in humans. Lysosomal enzymes may show preferential accumulation in particular tissues. Preferred medical indications for diagnostic uses include, for example, any condition associated with a target organ (e.g., lung, liver, kidney, spleen) or target tissue (e.g., bone, muscle) of interest.

The subject methods find use in the treatment of a variety of different disease conditions. In certain embodiments, of particular interest is the use of the subject methods in disease conditions where a lysosomal enzyme having desired activity has been previously identified, but in which the lysosomal enzyme is not adequately delivered to the target site, area or compartment to produce a fully satisfactory therapeutic result. With such lysosomal enzymes, the subject methods of producing highly phosphorylated lysosomal enzymes can be used to enhance the therapeutic efficacy and therapeutic index of active lysosomal enzyme.

Treatment is meant to encompass any beneficial outcome to a subject associated with administration of a lysosomal enzyme including a reduced likelihood of acquiring a disease, prevention of a disease, slowing, stopping or reversing, the progression of a disease or an amelioration of the symptoms associated with the disease condition afflicting the host, where amelioration or benefit is used in a broad sense to refer to at least a reduction in the magnitude of a parameter, e.g., symptom, associated with the pathological condition being treated, such as inflammation and pain associated therewith. As such, treatment also includes situations where the pathological condition, or at least symptoms associated therewith, are completely inhibited, e.g., prevented from happening, or stopped, e.g., terminated, such that the host no longer suffers from the pathological condition, or at least no longer suffers from the symptoms that characterize the pathological condition.

A variety of hosts or subjects are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Having now generally described the invention, the same may be more readily understood through the following reference to the following examples which provide exemplary protocols for the production, and purification of highly phosphorylated lysosomal enzymes and their use in the treatment of lysosomal storage diseases. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXAMPLES

### Example I

### G71 Cell Line Development

The objective was to produce a recombinant, highly phosphorylated lysosomal enzyme that is useful therapeutically at low doses. A cell line was developed that provides improved phosphorylation levels of recombinantly expressed lysosomal enzymes.

G71 cells (from Rockford K. Draper) were derived directly from CHO-K1 (ATCC CCL-61).

The adherent G71 cells were maintained at 34° C. in BioWhittaker UltraCHO medium supplemented with 2.5% fetal calf serum, 2 mM glutamine, gentamycin and amphotericin.

To allow easier use of cell lines for protein production, the adherent G71 cells may be pre-adapted to serum-free growth medium using a protocol for adapting anchorage-dependent, serum-dependent mammalian cells to high density serum-free suspension culture (Sinacore et al., Mol. Biotechnol. 15(3):249-257, 2000), resulting in the serum-free suspension culture adapted cell line, G71S. Alternatively, adherent G71 cells, after being stably transfected as described infra, may be adapted to serum-free suspension culture following procedures known in the art and basically as outlined in Sinacore et al., (2000), supra.

### Example II

### Human Acid Alpha-Glucosidase (GAA) Expression Vectors

The objective was to produce a recombinant, human acid alpha-glucosidase (hGAA). Mammalian expression vectors were generated encoding hGAA.

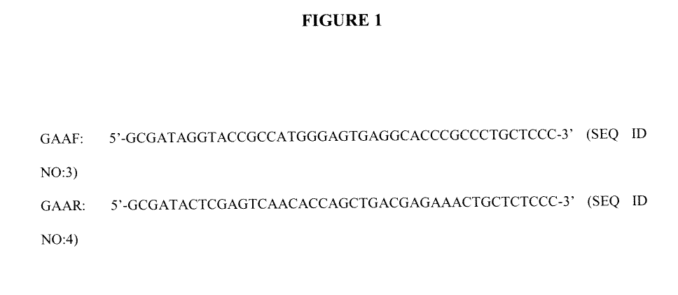

Human acid alpha-glucosidase (hGAA) cDNA was amplified from human liver mRNA (Clontech 6510-1) by high-stringency PCR using the primers designated GAAF and GAAR (SEQ ID NO:3 and SEQ ID NO:4, respectively) (FIG. 1).

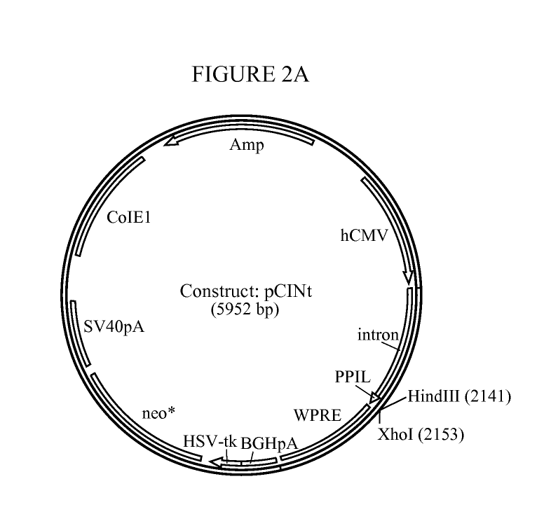

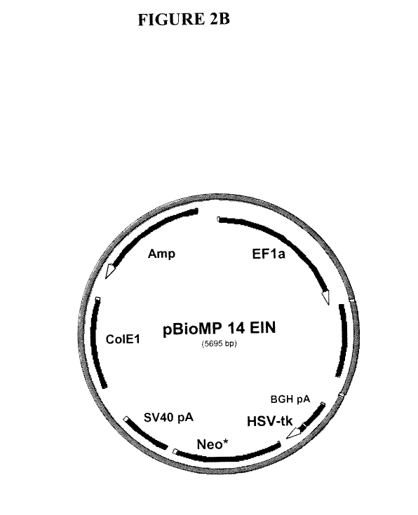

The amplified hGAA cDNA sequence was subcloned using flanking KpnI and XhoI sites into mammalian expression vector pCINt (BioMarin) (FIG. 2). This expression vector contained the human CMV enhancer-promoter linked to the rabbit β-globin IVS2 intron and the multiple cloning site from pcDNA3.1 (+) (Invitrogen, Carlsbad, Calif.). Efficient transcript termination was ensured by the bovine growth hormone poly-adenylation signal. The selection marker was a neomycin phosphotransferase gene that carries a point mutation to decrease enzyme efficiency. The attenuated marker was further handicapped with the weak HSV-tk promoter. The resultant rhGAA expression plasmid was named pCINt-GAA.

Alternatively, amplified hGAA cDNA sequence was subcloned into the multiple cloning site of the mammalian expression vector pBMP 14 EIN (BioMarin) (FIG. 2). This expression vector contained the human elongation factor-1α promoter linked to the rabbit β-globin IVS2 intron and the multiple cloning site from pcDNA3.1 (+) (Invitrogen, Carlsbad, Calif.). Efficient transcript termination was ensured by the bovine growth hormone poly-adenylation signal. The selection marker was a neomycin phosphotransferase gene that carries a point mutation to decrease enzyme efficiency. The attenuated marker was further handicapped with the weak HSV-tk promoter. The resultant rhGAA expression plasmid was named pBMP14-GAA.

In certain constructs, the hGAA cDNA was modified to replace the pre-prosequence of hGAA (MGVRHPPCSHRLLAVCALVSLATAALLG) (SEQ ID NO:5) with the presequence of human arylsulfatase B (MGPRGAASLPRGPGPRRLLLPVVPLLLLLLLAPPG) (SEQ ID NO:6). The resultant plasmid was named pBMP14-asbGAA.

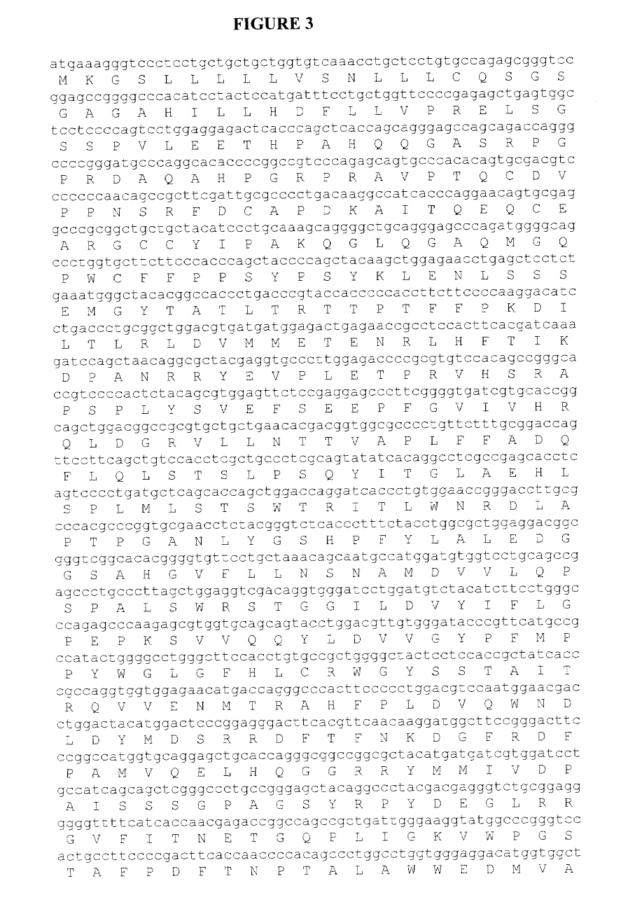

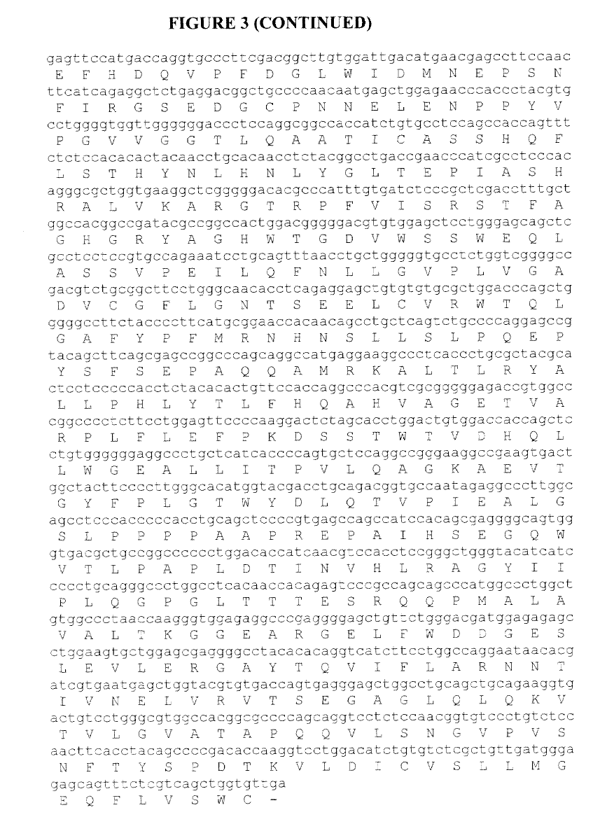

In certain constructs, the hGAA cDNA was modified to replace the pre-prosequence of hGAA with the presequence of human prolactin (MKGSLLLLLVSNLLLCQS) (SEQ ID NO:7) followed by a portion of human arylsulfatase B (GSGAGA) (SEQ ID NO:8). The nucleotide and amino acid sequences of this recombinant hGAA inserted into the expression vector plasmids is shown in FIG. 3 (SEQ ID NO:9 and SEQ ID NO:10, respectively). The resultant plasmid was named pBMP14-proGAA.

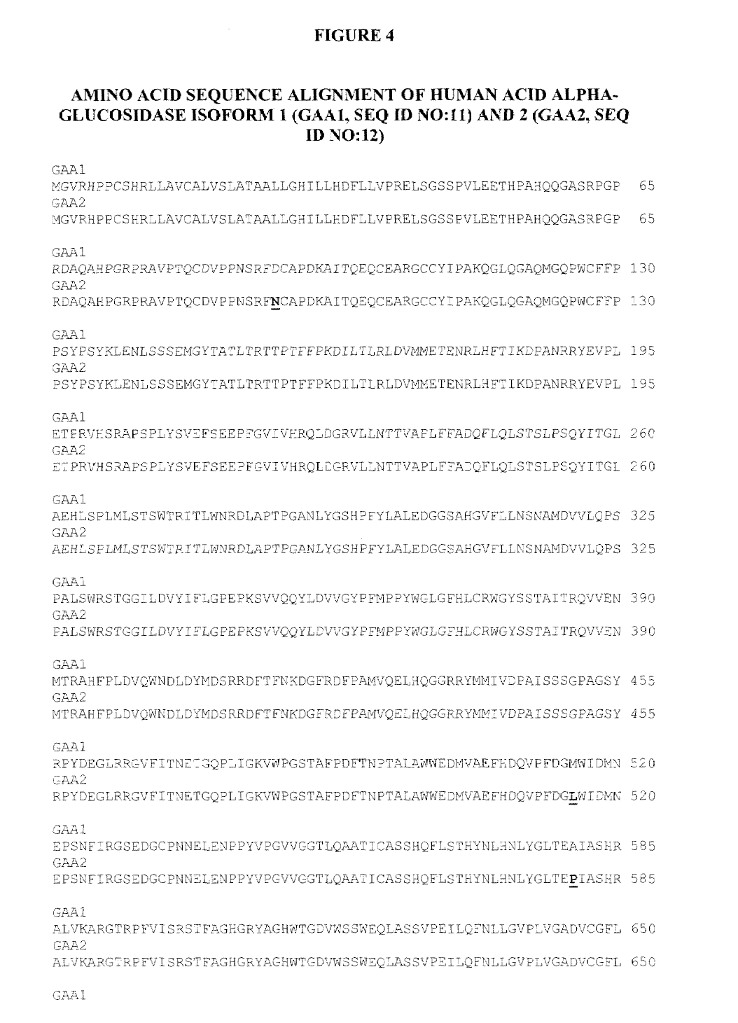

Human GAA exists in at least three different isoforms, the preponderant allele GAA1, GAA2 and GAA4. There are no known functional differences between these GAA isoforms. The GAA1 isoform is identical to the human acid alpha-glucosidase sequence in GenBank (Accession Number CAA687631), whereas the GAA2 isoform has amino acid substitutions at positions 91 (D91N), 515 (M515 L) and 580 (A580P). An amino acid sequence alignment of GAA1 (top, SEQ ID NO:11) and GAA2 (bottom, SEQ ID NO:12) is shown in FIG. 4.

The GAA2 isoform was inserted into the pCINt-GAA and pBMP14-GAA plasmids. The GAA2 isoform was mutagenized to the GAA1 isoform using the Stratagene QuikChange kit and appropriate primers.

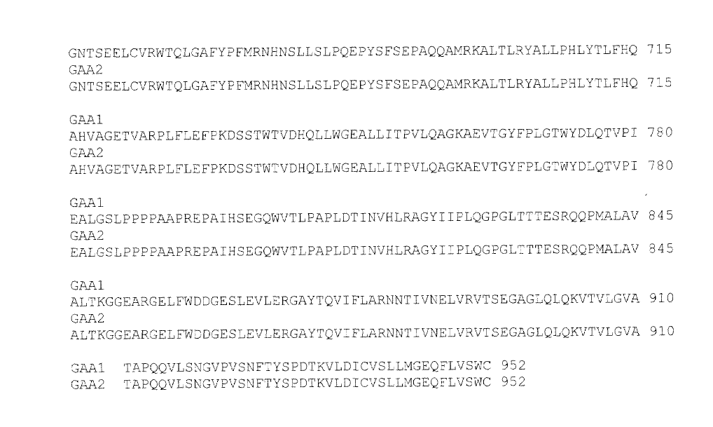

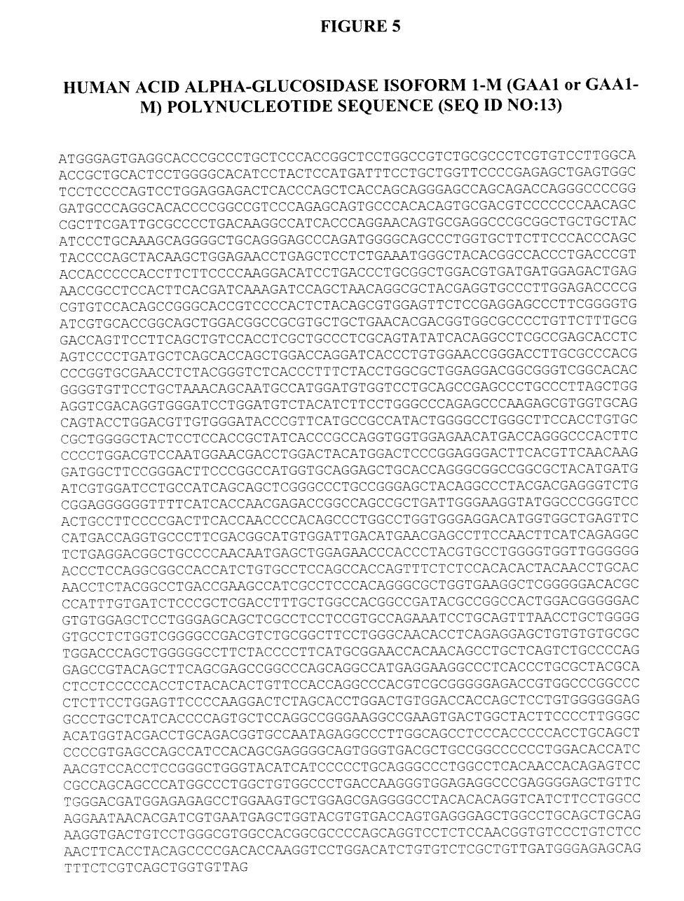

The nucleotide sequence of the recombinant hGAA isoform GAA1, also known as GAA1-M (amino acid methionine at position 515 of hGAA), which was inserted into the expression vectors of the invention, is depicted in FIG. 5 (SEQ ID NO:13). The amino acid sequence of the recombinant hGAA isoform GAA1 or GAA1-M, which is encoded by the expression vectors of the invention, is shown in FIG. 4 (SEQ ID NO:11).

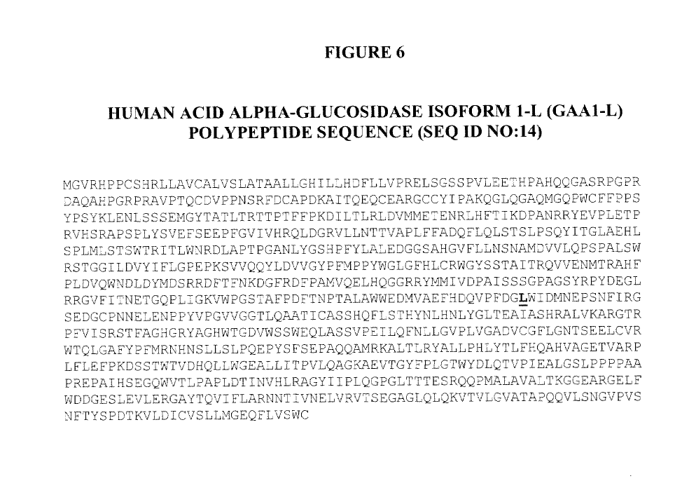

The GAA1 or GAA1-M isoform was mutagenized to a GAA1-L isoform (amino acid at position 515 changed from methionine to leucine) using the Stratagene QuikChange kit and appropriate primers. The amino acid sequence of the recombinant hGAA isoform GAA 1-L, which was inserted into the expression vectors of the invention, is depicted in FIG. 6 (SEQ ID NO:14).

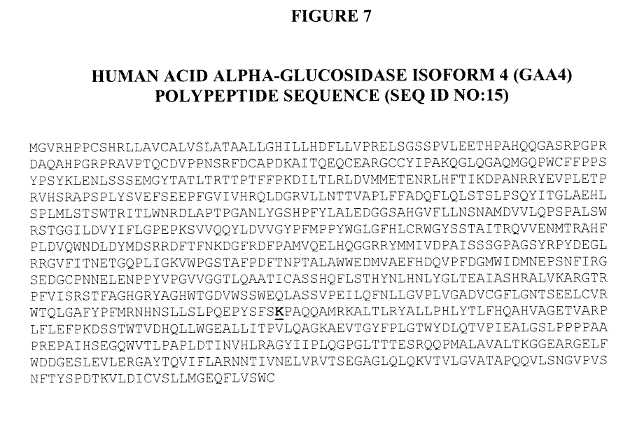

The GAA1 or GAA1-M isoform was mutagenized to a GAA4 isoform (amino acid at position 689 changed from glutamic acid to lysine) using the Stratagene QuikChange kit and appropriate primers. The amino acid sequence of the recombinant hGAA isoform GAA4, which was inserted into the expression vectors of the invention, is depicted in FIG. 7 (SEQ ID NO:15).

Table 2 shows the amino acid residues at the indicated positions in different human acid alpha-glucosidase (GAA) isoforms.

TABLE 2
Amino Acid Sequences in Human Acid
Alpha-Glucosidase (GAA) Isoforms

| | SEQ ID | | | | |
|---|---|---|---|---|---|
| Isoform | NO: | AA 91 | AA 515 | AA 580 | AA 689 |
| GAA1 (GAA1-M) | 11 | D | M | A | E |
| GAA1-L | 14 | D | L | A | E |
| GAA2 | 12 | N | L | P | E |
| GAA4 | 15 | D | M | A | K |

### Example III

### Development of G71 Cell Lines Expressing Recombinant Human Acid Alpha-Glucosidase (GAA)

The objective was to obtain highly phosphorylated recombinant human acid alpha-glucosidase (rhGAA). Expression vectors encoding rhGAA were transfected into G71 cells.

In a first series of transfections, G71 cells were transfected with linearized pCINt-based rhGAA expression plasmids. Transfections were performed by lipofection with Cytofectene (BioRad) using manufacturer's protocols. Stably transfected cells were selected on UltraCHO (BioWhittaker) supplemented with glutamine, gentamycin, amphotericin and 200 μg/mL G418 (Mediatech). Individual clones were obtained by limiting dilution and screened for activity by enzyme assay (see below). Following selection, stably transfected cells were transferred to JRH302 medium supplemented with glutamine, gentamycin, amphotericin and G418 for expression studies. G71 cells are grown at 34° C. in 5% CO₂ and induced at 39° C. in 5% CO₂ in the presence of 3 μM FeSO₄. Control BHK cells are grown at 37° C. in 5% CO₂.

After a first round of subcloning of transfectants, four rhGAA positive cell lines were selected using the fluorescent substrate, 4MU-alpha-glucoside, and enzyme produced by the cells (Reuser et al., Am. J. Hum. Genet. 30:132-43, 1978). This substrate yields 4-methylumbelliferone (4MU) after hydrolysis, which is detectable by a characteristic blue fluorescence when illuminated with UV-light (approximately 366 nm). These positive G71 clones were designated CIN4, 5, 6 and 11. Cell-specific productivity ranged from 1.8 and 4.6 pg cell of product. The four CIN cell lines were analyzed for enzyme production in spinners with microcarriers.

For comparison, dihydrofolate reductase deficient CHO cells, DUXB11, overexpressing rhGAA were prepared by similar means. The highest producing DUXB11 clone, 3.1.36, was selected for further studies.

In a second series of transfections, G71 cells were transfected with linearized pBMP14-based rhGAA expression plasmids. Transfections were performed in adherent G71 and CHOK1 cell lines as described above. Pools of transfectants were subcloned by limiting dilution cloning at 0.5, 1.0 and 1.5 cells/well in 96-well format. The activity was determined by the 4MU alpha-glucoside hydrolysis assay for approximately 300 clones. The clones with the highest activity were subsequently transferred and expanded.

Twenty clones from each pool of transfectants were analyzed at the T-25 flask level for activity and cell growth. CHOK1 clones transfected with GAA1 were B6 and B7. A G71 clone transfected with GAA1-L was 1B8. G71 clones transfected with GAA1-M were C3, D7 and F5. These clones were further evaluated for cell growth and production.

### Example IV

### Culture of Recombinant Human Acid Alpha-Glucosidase Expressing G71 Cells

The objective was to measure recombinant human acid alpha-glucosidase (rhGAA) production from the G71 transfectants. Cell lines were further assessed for rhGAA enzyme production in cell culture.

G71 transfected cell lines were cultured in JRH Excell 302 medium supplemented with 2 mM glutamine and 5% fetal calf serum. Cells were seeded onto Cytopore microcarriers (Pharmacia/Amersham) and grown in 200 mL spinner flasks. Serum was removed by dilution over the course of a week until BSA was undetectable by ELISA. The four CIN lines from the first series of transfections were analyzed for rhGAA production. CIN11 titer was the best producer at approximately 4 mg/L/day. DUXB11 3.1.36 titer was approximately 1 mg/L/day.

The best candidate from this screen, CIN11 (also known as G71GAA2) was scaled-up to bioreactor for production of pre-clinical material and analyzed for rhGAA production.

The GAA1-expressing G71 and CHOK1 cell lines from the second series of transfections were cultured as described above and analyzed for rhGAA production. Clones 1B8, B6, B7, C3, D7 and F5 were scaled-up to bioreactor for production of pre-clinical material and analyzed for rhGAA production.

The GAA1-expressing G71 cell lines were adapted for growth in suspension. Cells were suspended in JRH Excell 302 medium containing 5% FBS, and then sequentially through 2.5%, 1% or no FBS in the presence of G418, followed by removal of G418. The G71 suspension clones were adapted to growth in CD-CHO medium (Invitrogen) by resuspending cells in a 50:50 mixture of JRH Excell 302:CD-CHO medium, and after two high viability passages, cells were resuspended into 100% CD-CHO medium. Clones C3-S, D7-S and F5-S were scaled-up to bioreactor for production of pre-clinical material and analyzed for rhGAA production.

### Example V

### Purification of Recombinant Human Acid Alpha-Glucosidase

The objective was to obtain a large quantity of recombinant human GAA (rhGAA). Transfected G71 cells expressing rhGAA were grown under bioreactor culture conditions on microcarrier beads or in suspension and rhGAA enzyme was purified from the culture medium.

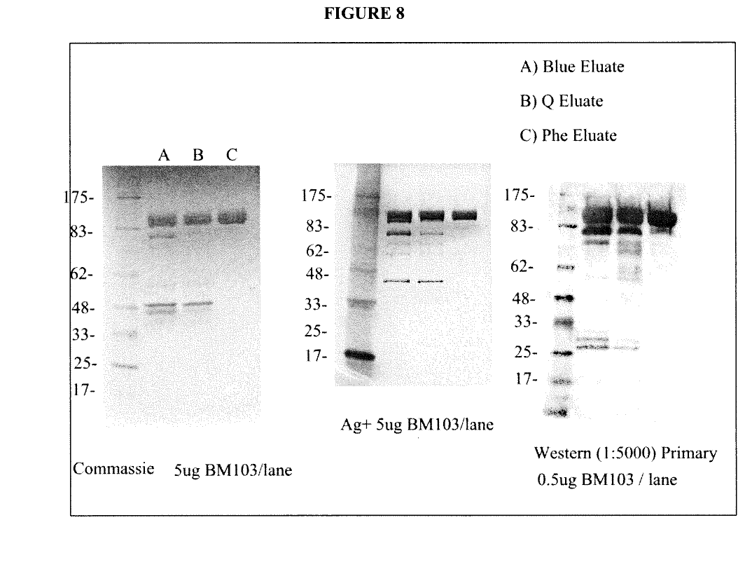

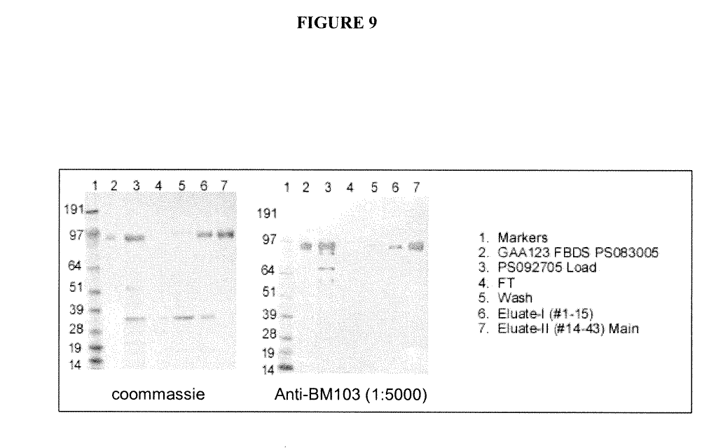

For enzymes from the first series of transfections, including rhGAA from CIN4, 5, 6 and 11, or from the second series of transfections, from 1B8, dia-filtered cell harvest medium was pH adjusted to 5.5, stored, adjusted to pH 4.5 and stored for 4 days at 4° C. Material was then re-filtered to remove precipitate. Yield was >90% for this step. Filtrate was then loaded onto Blue-Sepharose (Pharmacia/Amersham), washed with 20 mM acetate/phosphate, 50 mM NaCl, pH 4.5 and eluted with 20 mM acetate/phosphate, 50 mM NaCl, pH 5.9. Yield for this step was >70%. Eluate was then loaded to Q-Sepharose (Pharmacia/Amersham), washed with 10 mM histidine, pH 6.0, 70 mM NaCl and eluted with 10 mM histidine, pH 6.0, 165 mM NaCl. Yield for this step was >50%. Eluate was salt and pH adjusted to 1.3M NaCl and 5.0, respectively, loaded to Phenyl-Sepharose (Pharmacia Amersham) and gradient eluted with 1.3M to 0.5 M NaCl. Final purity of the rhGAA from a G71 transfectant grown on microcarrier beads (CIN11) was greater than 98% as assessed by SDS-PAGE followed by Coomassie stain, silver stain and Western blot (FIG. 8). Final purity of the rhGAA from a G71 transfectant grown in suspension (e.g., 1B8) was at least 95% as assessed by SDS-PAGE followed by Coomassie stain and Western blot (FIG. 9). Similar results were obtained for rhGAA from other G71 or G71-S transfectants using this three-column purification procedure.

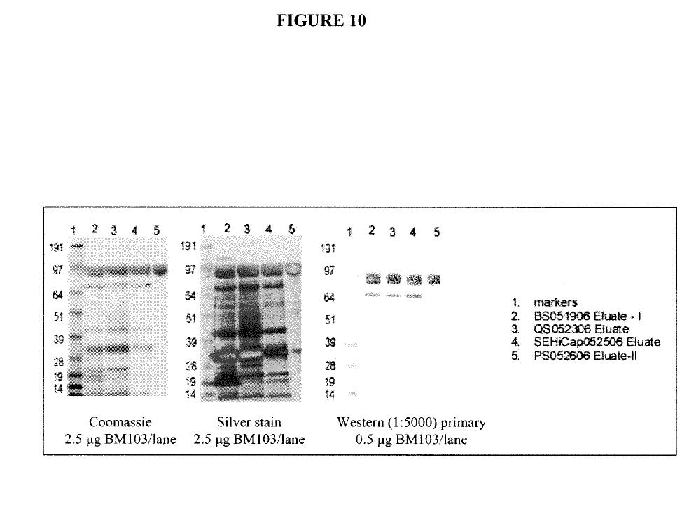

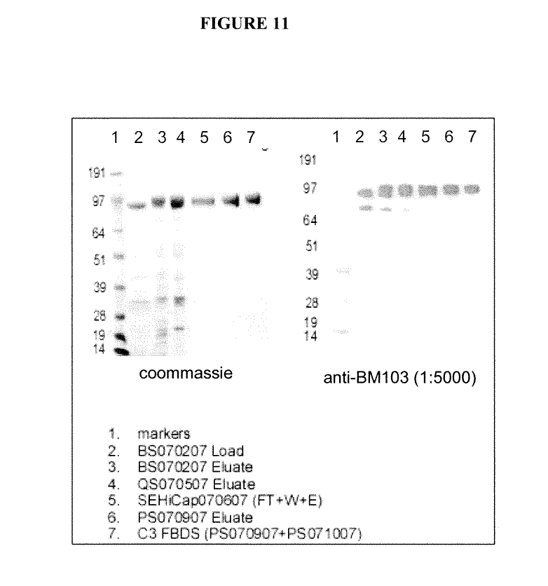

For enzymes from the second series of transfections, including 1B8, C3, D7, F5 and F6, ultrafiltered cell harvest medium was pH adjusted to pH 4.5 and stored at 4° C. Material was then re-filtered to remove precipitate. Yield was >90% for this step. Filtrate was then loaded onto Blue-Sepharose, washed with 20 mM acetate/phosphate, 50 mM NaCl, pH 4.5 and eluted with 20 mM acetate/phosphate, 50 mM NaCl, pH 5.9. Yield for this step was >70%. Eluate was then loaded onto Q-Sepharose, washed with 10 mM histidine, pH 6.0, 70 mM NaCl and eluted with 10 mM histidine, pH 6.0, 165 mM NaCl. Yield for this step was >50%. Eluate was then loaded onto SE Hi-CAP, washed with 10 mM acetate, 1 mM phosphate, 5 mM NaCl, pH 5.0 and eluted with a gradient of 5-300 mM NaCl in 10 mM acetate, 1 mM phosphate, pH 5.0. Yield for this step was ˜85%. The lysosomal enzyme, i.e., acid-alpha-glucosidase, was present in the flow-through, wash and early part of the salt elution gradient. Fractions containing lysosomal enzyme were pooled, adjusted to 2 M NaCl and then loaded onto Phenyl-Sepharose and gradient eluted with 1.3M to 0.5 M NaCl. Yield for this step was ˜80%. Eluate was formulated in pH 5.8 buffer. Final purity of the rhGAA from a G71 transfectant grown on microcarrier beads (e.g., F5) at least 95% as assessed by SDS-PAGE followed by Coomassie stain, silver stain and Western blot (FIG. 10). Final purity of the rhGAA from a G71 transfectant grown in suspension (e.g., C3) at least 95% as assessed by SDS-PAGE followed by Coomassie stain, silver stain and Western blot (FIG. 11). Similar results were obtained for rhGAA from other G71 or G71-S transfectants using this four-column purification procedure.

These assays indicate that the protocols described above for making recombinant lysosomal enzyme provide efficient methods for production of large quantities of highly purified enzyme, in particular, rhGAA.

### Example VI

### Analysis of Recombinant Human Acid Alpha-Glucosidase

The G71 cell line produces lysosomal enzymes with greater levels of high mannose phosphorylation than is noted in an average mammalian cell line, and a low level of unphosphorylated high-mannose oligosaccharides. A molecule comprising a low level of unphosphorylated high-mannose oligosaccharides, as defined herein, is compared to molecules obtained in U.S. Pat. No. 6,537,785 (Canfield), which do not comprise complex oligosaccharides, and exhibit only high mannose oligosaccharides.

To determine levels of unphosphorylated high-mannose, one of skill in the art can use exoglycosidase sequencing of released oligosaccharides (“FACE sequencing”), to pinpoint the percentages of unphosphorylated high-mannose oligosaccharide chains. On a normal lot-release FACE profiling gel, unphosphorylated high mannose co-migrates with particular complex oligosaccharides (for example, oligomannose 6 and fully sialylated biantennary complex). Unphosphorylated high mannose is then differentiated from the other oligosaccharides by enzymatic sequencing.

To determine if purified, recombinant human acid alpha-glucosidase (rhGAA) exhibits increased phosphorylation, the level of mannose-6-phosphate on the protein and enzyme binding to the mannose 6-phosphate receptor were determined.

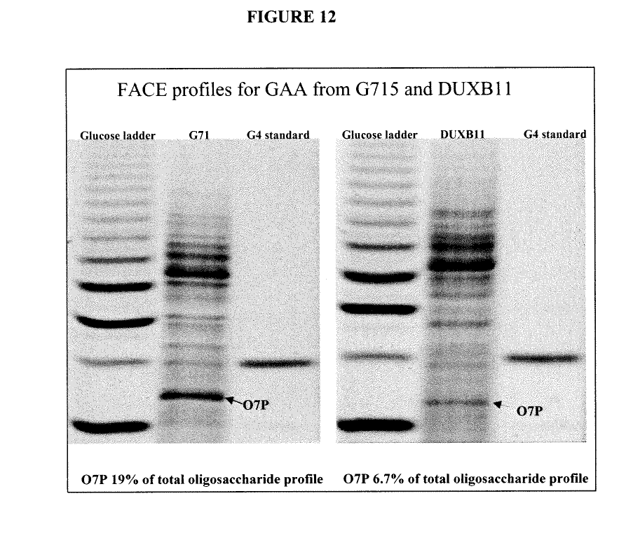

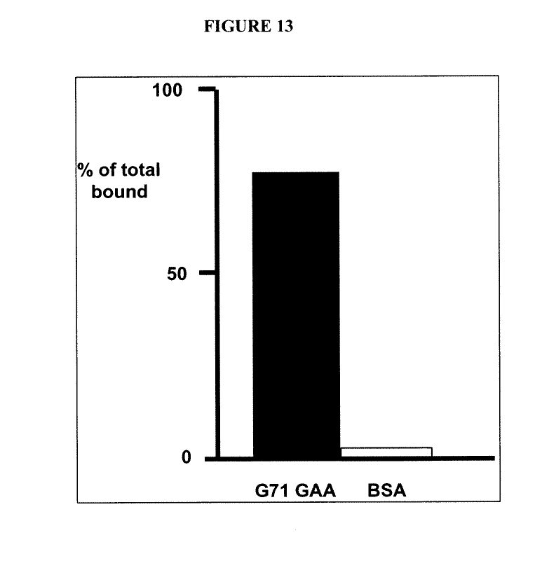

Purified, recombinant enzyme from the two transfected cell lines, G71 CIN11 and DUXB11, was analyzed by fluorescence assisted carbohydrate electrophoresis (FACE) and by chromatography on MPR-Sepharose resin. The relative intensity of the oligomannose 7 bis-phosphate (O7P) band on FACE (Hague et al., Electrophoresis 19(15):2612-2620, 1998) and the percent activity retained on the MPR column (Cacia et al., Biochemistry 37(43):15154-15161, 1998) gave reliable measures of phosphorylation level per mole of protein. A FACE comparison of material prepared from the G71 and DUXB11 lines showed that approximately 19.6% of the total G71 rhGAA oligosaccharide is O7P while only 6.7% of DUXB11 rhGAA is O7P (FIG. 12). This assay also demonstrated that approximately 75% of total binding activity to mannose 6 phosphate receptor column was attributed to G71 rhGAA (FIG. 12). Relative retention of enzyme analyzed by MPR column also demonstrated that approximately 75% of rhGAA bound to the receptor whereas binding of control protein was negligible (FIG. 13).

These results showed that the levels of mannose 6-phosphorylation was approximately 3-times higher in rhGAA enzyme produced by G71 cells than other CHO cell lines. Thus, G71 cells transfected with lysosomal enzyme efficiently produce highly phosphorylated enzyme, leading to an increased level of high mannose residues on these enzymes, which may lead to increased uptake by MPR on cells.

The level of mannose 6-phosphorylation on lysosomal enzymes from G71 cells expressing rhGAA was also determined by N-linked carbohydrate profiling by capillary electrophoresis as described in Ma et al., Anal. Chem. 71(22):5185-5192, 1999. The method used PNGase F to cleave N-linked oligosaccharides. The cleaved oligosaccharides were isolated and derivatized with fluorescent dye, and applied to a G10 spin column to remove excess dye. The purified, fluorescently labeled oligosaccharides were separated electrophoretically and peaks subsequently quantified using the MDQ-CE software (32 Karat Ver. 7.0).

Using this method, between 15% and 18% of the total oligosaccharides contained bis-phosphorylated mannose7 (BisPMan7) in rhGAA derived from G71 derivatives, including C3 grown in suspension in JRH-302 or CD-CHO medium, and F5 grown on microcarrier culture. This percentage of BisPMan7 oligosaccharides, assuming that all of the 7 N-linked glycosylation sites were glycosylated, corresponded to between 1.05 to 1.26 moles BisPMan7 per mol of enzyme. The results are shown in Table 3.

TABLE 3
Characterization of rhGAA Produced in G71 Cells

| | | | Sp. | | Sialic | Kᵤₚₜₐₖₑ |
|---|---|---|---|---|---|---|
| G71 | | Titer | Activity | BisPMan7 | Acid | GM248 |
| Clone* | Media | (mg/mL) | (U/mg) | % by CE | (Dionex)^{#} | (nM) |
| B7 | JRH- | 2.7 | 6.1 | 14 | 9.8 | 1.6 |
| | 302 | | | | | |
| C3-S | CD- | 8.0 | 5.2 | 17 | 5.5 | 2.1 |
| | CHO | | | | | |
| C3-S | JRH- | 10.0 | 8.0 | 18 | 5.9 | 1.9 |
| | 302 | | | | | |
| F5 | JRH- | 15.0 | 7.2 | 15 | 3.5 | 2.2 |
| | 302 | | | | | |

*S signifies G71 transfectant adapted to suspension culture.
^{#}Data is shown in mol of sialic acid/mol of protein using a Dionex instrument.

As shown in Table 3, the rhGAA from the G71 transfectant C3-S, which was adapted to growth in suspension in JRH-302 medium, was produced at a higher titer, with higher specific activity, higher level of phosphorylation, and lower level of sialic acid content than the rhGAA from the CHOK1 transfectant B7.

The ability of rhGAA from G71 cells in the second series of transfections to bind to the MPR was determined. The soluble extracellular domain of the bovine cation independent mannose-6-phosphate receptor (CI-MPR) was coupled to an Affigel-15 column using N-hydroxysuccinimide chemistry. Samples of rhGAA (6 μg) were bound to the CI-MPR column in a buffer of PBS pH 6.0/5 mM β-glycerophosphate/0.05% Tween-20/5 mM glucose-1-phosphate/0.02% sodium azide. Bound enzyme was subsequently eluted using a linear gradient of 0.5 mM mannose-6-phosphate in the same buffer without glucose-1-phosphate. Fractions were collected and assayed for GAA enzyme activity using the 4MU-alpha-glucoside assay or by an ELISA procedure. Using this assay, approximately 70% of rhGAA from the G71 transfectant F5 was able to bind to the immobilized MPR. Similar data was obtained for other rhGAA enzymes from G71 cells in the second series of transfections.

The percentage of rhGAA from the G71 transfectant F5 that was able to bind to the immobilized MPR in this assay, 70%, compared favorably to the 30%-40% of rhGAA produced in CHO cells that was able to bind to a similar CI-MPR column, as reported in Zhu et al., J. Biol. Chem. 279:50336-50341, 2004 and Zhu et al., Biochem. J. 389:619-628, 2005.

In summary, rhGAA has been produced in G71 cells with a high titer and having a high specific activity, high level of phosphorylation and ability to bind to the MPR in vitro.

### Example VII

### Uptake of Recombinant Human Acid Alpha-Glucosidase into Pompe Fibroblasts

The objective was to determine if the purified recombinant human acid alpha-glucosidase (rhGAA) protein binds efficiently to the MPR on cells. Cells obtained from patients with the lysosomal storage disorder Pompe disease were assessed for their ability to bind recombinant, highly phosphorylated rhGAA.

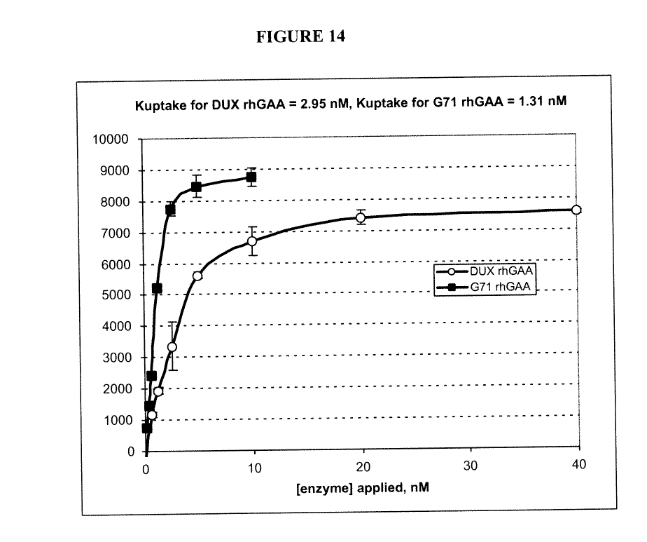

The uptake of rhGAA enzyme from the first series of G71 transfections was investigated in GM244 Pompe patient fibroblasts. Cells were seeded and grown to confluence in 12-well plates. On the day of the experiment, cells were fed with fresh medium containing 4 mM glucose and varying concentrations of either G71 rhGAA or DUXB11 rhGAA. Cells were incubated for 4 hours, rinsed with PBS and lysed by freeze-thaw. GAA enzyme activity was then measured using 4MU-alpha-glucoside using published methods. The 4MU-alpha-glucoside assay demonstrated that the rate of enzyme uptake (Kᵤₚₜₐₖₑ) for DUXB11 rhGAA was 2.95 nM and the Kᵤₚₜₐₖₑ for G71 rhGAA was 1.31 nM (approximately 2.25 times more efficient that the DXB11 rhGAA) (FIG. 14).

This result demonstrated that phosphorylated high-mannose oligosaccharide on the G71-derived acid alpha-glucosidase bound to the MPR with an affinity similar to that seen for other properly phosphorylated lysosomal enzymes (Sando et al., Cell 12:619-27, 1977). This affinity for the MPR exceeded that for acid alpha-glucosidase made in DUXB11.

The uptake of rhGAA enzyme from the second series of G71 transfections, either from clone F5 or C3-S, was investigated in confluent GM248 Pompe fibroblasts. Cells were exposed to a dilution series of each enzyme in serum-free uptake medium (DMEM/25 mH HEPES, pH 7.5/2 mM glutamine/0.5 mg/mL BSA) for 4 hours. After exposure, cells were washed with PBS and released from the plate with trypsin/EDTA digestion. Cells were collected, washed with PBS and lysed by freezing overnight in phosphate-citrate buffer pH 4.0/0.1% Triton-X100. Crude extracts were thawed and centrifuged to separate the soluble and insoluble phases, and then the soluble lysate was analyzed for enzyme activity using the 4MU-alpha-glucoside substrate assay.

As shown in Table 3 above, this assay demonstrated that the rate of enzyme uptake (Kᵤₚₜₐₖₑ) for rhGAA from G71 cells was 1.9-2.2 nM. These results suggested that the highly phosphorylated rhGAA produced in G71 cells bound to the MPR on cells with high affinity and was taken up into the cells in an active form.

### Example VIII

### Measurement of Specific Uptake of Recombinant Human Acid-Alpha-Glucosidase into Enzyme-Deficient Patient Fibroblasts with Concomitant Clearance of Stored Glycogen

The ability of the recombinant human acid alpha-glucosidase (rhGAA) enzyme to clear glycogen stored in cells is assessed.

Fibroblasts from patients symptomatic of a glycogen storage disorder are seeded and grown to confluence in 12-well plates. On the day of the experiment, cells are fed with fresh medium containing 4 mM glucose. Cells are also supplemented with rhGAA in the presence or absence of 10 mM mannose 6-phosphate. Cells are harvested each day for 4 days. After rinsing with PBS, cells are lysed by freeze-thaw. Stored glycogen is assayed by boiling the lysate, precipitation with 80% ethanol, digestion with Aspergillis niger glucosidase and glucose assay (Van Hove et al., Proc. Natl. Acad. Sci. USA 93:65-70, 1996). Stored glycogen values are normalized to the protein content of the cell lysates.

It is expected that cells receiving G71 rhGAA clear stored glycogen more efficiently than cells which are treated with enzyme produced by other recombinant methods or control protein. Ability of G71 rhGAA treated cells to clear glycogen at levels comparable to cells from normal donors indicates that the G71 produced lysosomal enzyme is as effective as native rhGAA enzyme in relieving symptoms of Pompe disease.

### Example IX

### Effect of Recombinant Human Acid-Alpha-Glucosidase in a Mouse Model of Pompe Disease

The effects of the highly phosphorylated human lysosomal enzymes of the invention, e.g., recombinant human acid alpha-glucosidase (rhGAA), in mice deficient in GAA (i.e., mouse model of Pompe disease) were studied.

The rhGAA enzyme from G71 clone C3 was expressed in G71 cells, purified, and administered into GAA deficient (Pompe) mice.

At least three mouse models of GAA deficiency (i.e., Pompe disease) have been described (Raben et al., J. Biol. Chem. 273(30): 19086-92, 1998; Bijvoet et al., Hum. Mol. Genet. 7(1):53-62, 1998). The 6^{neo}/6^{neo} mice, which have markedly reduced mobility and strength by 3.5 weeks of age, recapitulate the critical features of the infantile and adult forms of Pompe disease, whereas the Δ6/Δ6 and Gaa(−/−) mice, despite displaying similar biochemical and pathological features as the 6^{neo}/6^{neo} mice, have unimpaired strength and mobility up to 6.5 months of age.

The effect of administration of rhGAA into 6^{neo}/6^{neo} mice, also referred to as GAA^{tm1Rabn/J} mice, on tissue glycogen clearance was investigated. GAA^{tm1Rabn/J} mice or wild-type mice of the background strain of about three months of age were administered vehicle or various doses of rhGAA by intravenous infusions in the tail vein over 30 min twice weekly for two weeks. Mice were sacrificed two days after the last injection. Details of this study are shown in Table 4.

TABLE 4
Design of Study to Determine Effect of
rhGAA in Mouse Model of Pompe Disease

| | No. | | Adminis- | Dose | | |
|---|---|---|---|---|---|---|
| Group | Animals | Strain | tered | (mg/kg) | Route | Frequency |
| 1 | 10 | Wild-type | Vehicle | 0 | IV | 2x/week, |
| | | | | | | 2 wks |
| 2 | 10 | GAA^{tm1Rabn/J} | Vehicle | 0 | IV | 2x/week, |
| | | | | | | 2 wks |
| 3 | 10 | GAA^{tm1Rabn/J} | rhGAA | 5 | IV | 2x/week, |
| | | | | | | 2 wks |
| 4 | 10 | GAA^{tm1Rabn/J} | rhGAA | 10 | IV | 2x/week, |
| | | | | | | 2 wks |
| 5 | 10 | GAA^{tm1Rabn/J} | rhGAA | 20 | IV | 2x/week, |
| | | | | | | 2 wks |

The following tissues were collected and analyzed for rhGAA activity and for glycogen clearance: heart (left ventricle); diaphragm; and skeletal muscle (quadriceps, triceps, psoas, soleus, plantaris).

Sera was collected from the mice at day 0 prior to the first injection, at day 7 before the third injection, and two days after the last injection when the mice were sacrificed.

Quantitative GAA enzyme activity assay in tissue homogenates. A method was used to measure GAA enzyme activity in Pompe mouse tissue with a limit of detection of 0.2 mU GAA/g tissue. Tissue was homogenized in 0.2 M sodium phosphate, 0.1 M citrate (pH 4.3) using the FastPrep homogenizer (MP Bio). GAA was captured on a plate coated with anti-GAA as described in Umapathysivam et al., Clin. Chem. 47:1378-1383, 2001. Enzyme activity was measured using a fluorometric assay as described in Kallwass et al., Mol. Genet. Metab. 90:449-452, 2007, and reported as mU GAA activity per gram of tissue. The results are shown in Table 5 and expressed as GAA activity in indicated tissues relative to those in the same tissues in normal mice treated with vehicle.

TABLE 5
Tissue rhGAA Enzyme Activity in
Mouse Model of Pompe Disease

| | Normal | 5 mg/kg | 10 mg/kg | 20 mg/kg | Pompe |
|---|---|---|---|---|---|
| Tissue | Mice# | rhGAA | rhGAA | rhGAA | Mice |
| Diaphragm | 100 ± 15 | 114 ± 77 | 165 ± 75 | 386 ± 145 | 6 ± 0 |
| Psoas | 100 ± 11 | 63 ± 35 | 175 ± 102 | 251 ± 86 | 8 ± 0 |
| Quadriceps | 100 ± 6 | 36 ± 18 | 126 ± 77 | 166 ± 80 | 7 ± 0 |
| Triceps | 100 ± 9 | 42 ± 18 | 139 ± 65 | 218 ± 106 | 7 ± 0 |

^{#}GAA activity in normal mice treated with vehicle was taken as 100.

The results showed a dose-dependent increase in GAA enzyme activity in Pompe mouse tissues upon administration of rhGAA. The levels of rhGAA enzyme activity achieved were greater than the levels of endogenous GAA enzyme activity in Pompe and normal mice.

Light microscopic analysis of tissue glycogen. High resolution light microscopy method was used to quantitate glycogen content in tissue as described in Lynch et al., J. Histochem. Cytochem. 53(1): 63-73, 2005. Tissues were cut into 1 mm³ pieces immediately after collection, immersion-fixed in 3% gluteraldehyde in 0.2 M cacodylic buffer, pH 7.3, post-fixed in 1% osmium tetroxide in 0.2M cacodylate buffer, pH 7.3, and then embedded in Epon-Araldite. Polymerized blocks were cut to 1 μm thick sections and mounted on charged slides. Slides were stained for Epon-Araldite, and then dried completely in a 60° C. oven and coverslipped with Acrytol without the use of xylene.

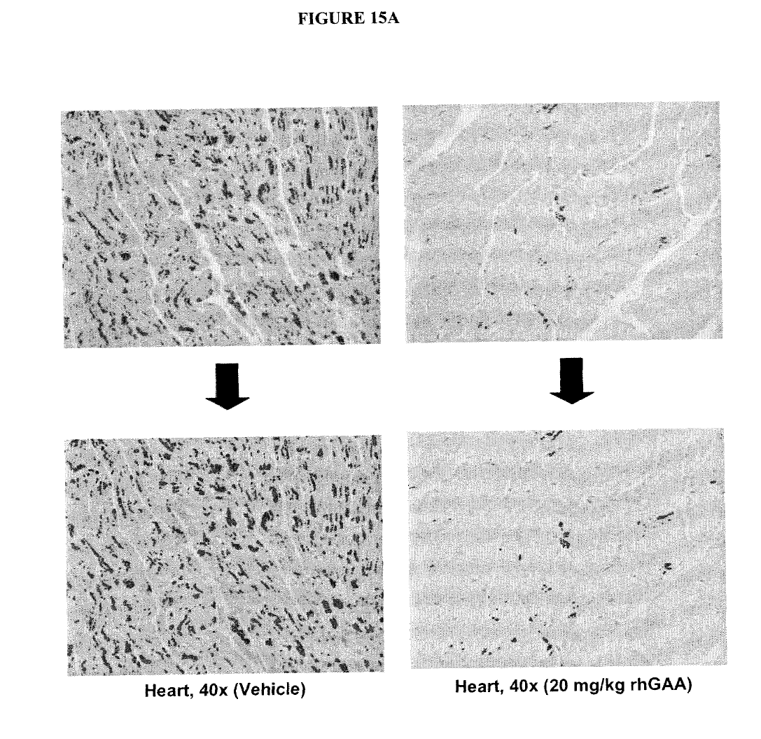

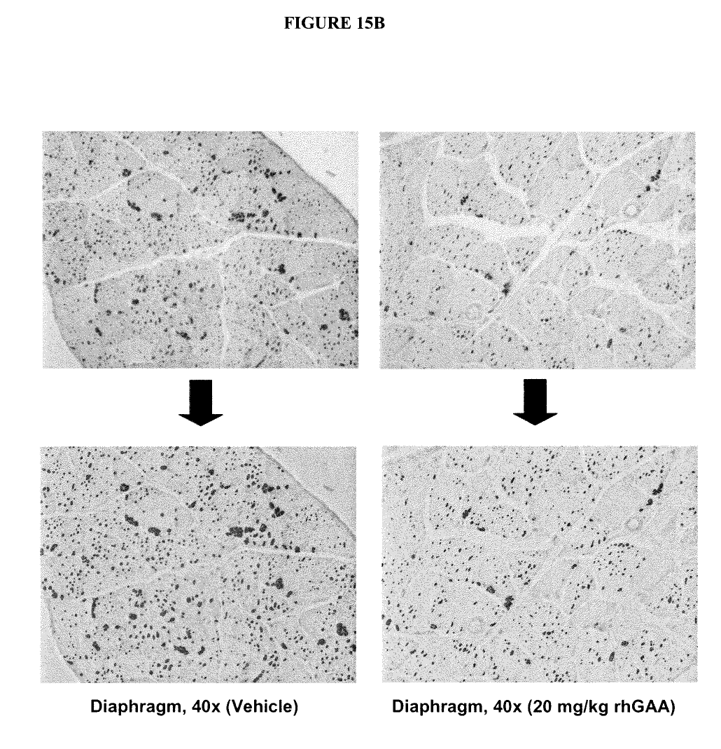

Stained sections were evaluated and photographed at the 40× magnification. ImagePro software was used to determine the percent area occupied by PAS stained glycogen in three fields for each tissue. The results for heart and diaphragm, comparing vehicle (left panels) and 20 mg/kg rhGAA (right panels) are shown in FIGS. 15A and 15B, respectively.

The results showed that rhGAA was able to significantly reduce the glycogen storage that accumulated in the heart and diaphragm in Pompe mice.

Quantitative measurement of glycogen in tissue homogenates. A quantitative glycogen determination method was used with a limit of detection of 0.02 mg glycogen/g tissue. Tissue was homogenized in 0.2 M sodium acetate, 0.5% NP-40 (pH 4.8) using the FastPrep homogenizer (MP Bio). Glycogen was detected by using two enzymatic steps as described in Zhu et al., J. Biol. Chem. 279:50336-50341, 2004, and reported as mg glycogen/g tissue. The results are shown in Table 6 and expressed as glycogen levels in indicated tissues relative to those in the same tissues in GAA^{tm1Rabn/J} mice treated with vehicle.

TABLE 6
rhGAA Reduces Tissue Glycogen in
Mouse Model of Pompe Disease^{#}

| | Normal | 5 mg/kg | 10 mg/kg | 20 mg/kg |
|---|---|---|---|---|
| Tissue | Mice | rhGAA | rhGAA | rhGAA |
| Heart | 2 ± 1** | 51 ± 14** | 31 ± 13** | 14 ± 7** |
| Diaphragm | 6 ± 4** | 87 ± 19 | 72 ± 14 | 51 ± 12** |
| Quadriceps | 7 ± 2** | 106 ± 16 | 100 ± 16 | 78 ± 12** |
| Triceps | 5 ± 3** | 86 ± 16* | 90 ± 15 | 74 ± 13** |
| Psoas | 8 ± 3** | 92 ± 13 | 91 ± 24 | 71 ± 13** |
| Plantaris | 14 ± 3** | 91 ± 18 | 82 ± 15* | 76 ± 12** |
| Soleus | 10 ± 4** | 79 ± 16** | 82 ± 18* | 54 ± 15** |

^{#}Glycogen levels in GAA^{tm1Rabn/J} mice treated with vehicle were taken as 100.
*p < 0.05;
**p < 0.01

The results showed that rhGAA was able, in a dose-dependent fashion, to reduce glycogen accumulation in all of the tissues examined, with the most striking effect being observed in the heart, as expected (Zhu et al., Biochem. J. 389:619-628, 2005). Thus, rhGAA produced in G71 cells was able to exert beneficial effects in vivo in a mouse model of Pompe disease.

Detection of antibodies to GAA in sera of Pompe mice after injection with rhGAA. A qualitative electrochemiluminescent assay (ECLA) was used to detect antibodies against rhGAA in mouse serum (White et al., U.S. application Ser. No. 11/724,983, filed Mar. 16, 2007). To determine the titer, serial 3-fold dilutions were prepared and the highest dilution factor yielding a positive result obtained (three times the serum dilution). The log₁₀ of this dilution factor was reported as the titer. The lowest reported titer was 1.5, which corresponded to log₁₀(30). Antibodies against rhGAA present in mouse serum were measured at three timepoints: predose (day 0), prior to the third dose (day 7), and at necropsy (day 13).

All predose serum samples were below the limit of detection, and only three of the day 7 serum samples generated a positive anti-GAA antibody titer. Less than half the mice had a detectable serum anti-GAA antibody titer at necropsy (day 13).

A second study was performed to further investigate the effect of administration of rhGAA on tissue glycogen clearance and on anti-rhGAA antibody formation in GAA^{tm1Rabn/J} mice. As in the first study, the rhGAA was expressed from the G71 clone C3 in G71 cells as described above. GAA^{tm1Rabn/J} mice or wild-type mice of the background strain (normal) of about five months of age were administered vehicle or various doses of rhGAA by bolus injection in the tail vein weekly for four weeks. Mice were sacrificed one week after the last injection. This study was modeled after the studies described in Zhu et al., J. Biol. Chem. 279:50336-50341, 2004 and Zhu et al., Biochem. J. 389:619-628, 2005, the disclosures of which are herein incorporated by reference in their entirety. Details of this study are shown in Table 7.

TABLE 7
Design of Study to Determine Effect of
rhGAA in Mouse Model of Pompe Disease

| | No. | | Adminis- | Dose | | |
|---|---|---|---|---|---|---|
| Group | Animals | Strain | tered | (mg/kg) | Route | Frequency |
| 1 | 10 | GAA^{tm1Rabn/J} | rhGAA | 5 | Bolus | 1x/week, |
| | | | | | | 4 wks |
| 2 | 10 | GAA^{tm1Rabn/J} | rhGAA | 10 | Bolus | 1x/week, |
| | | | | | | 4 wks |
| 3 | 10 | GAA^{tm1Rabn/J} | rhGAA | 20 | Bolus | 1x/week, |
| | | | | | | 4 wks |
| 4 | 10 | GAA^{tm1Rabn/J} | Vehicle | — | Bolus | 1x/week, |
| | | | | | | 4 wks |
| 5 | 10 | Normal | Vehicle | — | Bolus | 1x/week, |
| | | | | | | 4 wks |

The following tissues were collected and analyzed for rhGAA activity and for glycogen clearance: heart (left ventricle); diaphragm; and skeletal muscle (quadriceps, triceps, psoas, soleus, plantaris).

Sera was collected from the mice at day 0 prior to the first injection, at day 7 before the third injection, at day 14 before the fourth injection, and at day 29, after the last injection when the mice were sacrificed.

Quantitative measurement of glycogen in tissue homogenates. The quantitative glycogen determination method described above was used to measure the amount of glycogen per gram of tissue. The results are shown in Table 8 and expressed as glycogen levels in indicated tissues relative to those in the same tissues in GAA^{tm1Rabn/J} mice treated with vehicle.

TABLE 8
rhGAA Reduces Tissue Glycogen in
Mouse Model of Pompe Disease^{#}

| | Normal | 5 mg/kg | 10 mg/kg | 20 mg/kg |
|---|---|---|---|---|
| Tissue | Mice | rhGAA | rhGAA | rhGAA |
| Heart | 1 ± 1** | 40 ± 10** | 32 ± 14** | 10 ± 5** |
| Diaphragm | 4 ± 2** | 101 ± 23 | 74 ± 13** | 42 ± 15** |
| Quadriceps | 2 ± 1** | 42 ± 21** | 45 ± 15** | 31 ± 12** |
| Triceps | 5 ± 4** | 91 ± 68 | 92 ± 36 | 41 ± 14** |
| Psoas | 5 ± 2** | 84 ± 15 | 94 ± 18 | 57 ± 25** |
| Plantaris | 9 ± 3** | 82 ± 22* | 77 ± 12** | 46 ± 20** |
| Soleus | 5 ± 3** | 45 ± 16** | 54 ± 21** | 21 ± 12** |

^{#}Glycogen levels in GAA^{tm1Rabn/J} mice treated with vehicle were taken as 100.
*p < 0.05;
**p < 0.01

This second study showed that rhGAA caused a dose-dependent reduction in glycogen accumulation in all tissues examined, with the most striking effect in the heart, consistent with the first study.

It was also apparent that glycogen clearance in this second (five week) study was improved compared to the first (three week) study. Thus, rhGAA produced in G71 cells is able to exert beneficial effects in vivo in a mouse model of Pompe disease.

Detection of antibodies to GAA in sera of Pompe mice after injection with rhGAA. The qualitative ECLA described above was used to detect antibodies against rhGAA in mouse serum. The highest dilution factor that provided a positive result in the assay was the reported value (log₁₀ reported as titer).

No pre-dose serum samples were positive, whereas most serum samples obtained post-dose showed a detectable anti-GAA antibody titer. The median anti-GAA titer increased for all treatment groups (5, 10 and 20 mg/kg rhGAA) between day 14 and day 29. This increased anti-GAA titer, however, was not rhGAA dose-dependent, and no correlation was observed between the anti-GAA titers and pharmacodynamic activity (i.e., glycogen clearance). These findings showed that rhGAA induced an antibody response in Pompe mice, but that the presence of anti-GAA antibodies did not preclude efficacy in this model of Pompe disease.

### Example X

### Treatment of Patients with Pompe Disease with Recombinant Human Acid-Alpha-Glucosidase

Human patients manifesting a clinical phenotype of lysosomal enzyme deficiency, such as in patients with Pompe Disease with an acid alpha-glucosidase (GAA) level of less than 1% of normal in leukocytes and fibroblasts are contemplated for enzyme replacement therapy with the recombinant human acid alpha-glucosidase (rhGAA) enzyme produced according to the methods described herein. All Pompe Disease patients manifest some clinical evidence of muscular accumulation of glycogen with varying degrees of functional impairment. Efficacy is evaluated by measuring enhancements or stabilization in cardiac, pulmonary and motor function and/or determining the percentage of patients who die during the study or require ventilatory support. Assessment of liver size is also performed as this is the most widely accepted means for evaluating successful ERT in Pompe disease (Hoogerbrugge et al., Lancet 345:1398, 1995).

A randomized, double-blinded clinical trial of purified, highly phosphorylated rhGAA produced according to the methods described herein is conducted in patients with Pompe Disease, in particular the infantile-onset form of the disease, but may also be conducted in patients with the juvenile- and late-onset forms of the disease.

A pharmaceutical composition consisting of an enzyme composition comprising rhGAA is administered intravenously. The final dosage form of the fluid comprises rhGAA, normal saline, phosphate buffer at pH 5.8 and human albumin at 1 mg/ml, prepared in a bag of normal saline. A preferred composition comprises rhGAA in an amount ranging from 0.05-0.5 mg/mL or 12,500-50,000 units per mL; sodium chloride solution 150 mM; sodium phosphate buffer 10-50 mM, pH 5.8; human albumin 1 mg/mL. The composition may be in an intravenous bag of 50 to 250 mL. When administered to patients, the rhGAA is prepared by diluting the appropriate amount of drug solution in the intravenous bag with normal saline at room temperature. Patients may be pre-medicated with an anti-histamine prior to infusion to reduce the potential for infusion-related reactions.

The objectives of this study are to evaluate the safety, efficacy and pharmacokinetic profile of various doses of rhGAA as ERT in patients with Pompe Disease. Patients are randomized in a double-blind fashion to various dose groups, which may range from about 1 to 40 mg/kg rhGAA. Drug is administered at a frequency which may range from about once per week to once per month as an infusion over a period of time which may range from about 1-hour to 6-hours, depending on the dose of enzyme. The length of treatment may range from about 26 to 104 weeks or more. Enrollment in this study may be limited to those Pompe Disease patients having clinical signs of the disease, but not requiring ventilatory support at the time of entry into the study.

Measures of safety include, for example, complete chemistry panel, urinalysis, complete blood count with differential, ECG and tracking of adverse events. Immune response and infusion-associated reactions, complement activation and antibody formation are assessed for all patients. Safety can be assessed by comparing the adverse safety events among patient groups administered different doses of rhGAA.

Efficacy parameters include, for example, exercise tolerance/endurance (e.g., 6-minute or 12-minute walk tests, an expanded timed get up and go test, a 3-minute stair climb test and physical activity), respiratory capacity (e.g., pulmonary function tests such as forced vital capacity, forced expiratory time and forced expiratory volume for one second), joint range of motion (flexion, extension, rotation), functional status (e.g., Childhood Health Assessment Questionnaire, including assessment of pain and stiffness), levels of urinary glycogen and glycosaminoglycans, and changes in hepatomegaly, assessment of grip and pinch strength, visual acuity, cardiac function (e.g., ECG) and sleep apnea. Efficacy can be assessed by measuring cardiac, pulmonary and/or motor function according to established guidelines and comparing the results among patient groups administered different doses of rhGAA. Efficacy can also be assessed by comparing the percentage of patients who die or require ventilatory support during the study with an historical cohort of untreated Pompe patients of similar age and disease severity.

Pharmacokinetic evaluations are conducted during infusions periodically to measure rhGAA enzyme levels during and after infusions.

Based on the invention and examples disclosed herein, those skilled in the art will be able to develop other embodiments of the invention. The examples are not intended to limit the scope of the claims set out below in any way. Although the foregoing invention has been described in some detail by way of illustration and example for purposes of clarity of understanding, it will be readily apparent to those of ordinary skill in the art, in light of the teachings of this invention, that numerous modifications and variations in the invention as set forth above illustrative examples are expected to occur to those skilled in the art and may be made thereto without departing from the spirit or scope of the appended claims. Consequently only such limitations as appear in the appended claims should be placed on the invention.

What is claimed:

1. A recombinant human acid alpha-glucosidase (rhGAA) enzyme comprising an amino acid sequence at least 90% identical to the GAA amino acid sequence set out in SEQ ID NO: 2, produced by END3 complementation group CHO cells, wherein said rhGAA enzyme has at least about 0.7 bis-phosphorylated oligomannose chains per mole of rhGAA enzyme, and wherein said END3 complementation group CHO cell is a G71 cell line or derivative thereof.

2. The rhGAA enzyme of claim 1, wherein said rhGAA enzyme comprises an amino acid sequence at least 95% identical to the GAA amino acid sequence set out in SEQ ID NO: 2.

3. A method for producing the rhGAA enzyme of claim 1, comprising culturing Chinese Hamster Ovary (CHO)-derived END3 complementation group cells under conditions appropriate for growth of the cells and expression of the GAA enzyme.

4. A method for producing the rhGAA enzyme of claim 1, comprising the steps of:
(a) culturing Chinese Hamster Ovary (CHO)-derived END3 complementation group cells;
(b) preparing a mammalian expression vector encoding said rhGAA enzyme or variant thereof suitable for said END3 complementation group cells;
(c) transfecting said END3 complementation group cells with said expression vector;
(d) selecting and cloning a END3 complementation group cell transfectant; and
(e) optimizing cell culture process methods for manufacturing said END3 complementation group cell transfectant,
wherein said END3 complementation group cell is a G71 cell line or derivative thereof.

5. The method of claim 4, wherein said rhGAA enzyme comprises an amino acid sequence at least 95% identical to the GAA amino acid sequence set out in SEQ ID NO: 2.

6. A recombinant human acid alpha-glucosidase (rhGAA) enzyme produced by the method of any one of claims 3 to 5.

7. A pharmaceutical composition comprising the rhGAA enzyme of claim 1 or 2, and a pharmaceutically acceptable carrier, diluent or excipient.

8. A pharmaceutical composition comprising the rhGAA enzyme of claim 6, and a pharmaceutically acceptable carrier, diluent or excipient.

9. A method of treating a deficiency of acid alpha-glucosidase comprising administering to a subject in need a therapeutically effective amount of the rhGAA enzyme of claim 1 or 2.

10. The method of claim 9, wherein said subject has Pompe disease.

11. The method of claim 9, wherein the subject is human.

12. An END3 complementation group CHO cell line comprising an expression vector for recombinant human acid alpha-glucosidase (rhGAA) enzyme, wherein said rhGAA enzyme comprises an amino acid sequence at least 90% identical to the GAA amino acid sequence set out in SEQ ID NO: 2.

13. The END3 complementation group CHO cell line of claim 12, wherein the END3 complementation group cell line expresses and secretes rhGAA or a variant thereof.

14. The END3 complementation group CHO cell line of claim 13, wherein said rhGAA enzyme comprises an amino acid sequence at least 95% identical to the GAA amino acid sequence set out in SEQ ID NO: 2.
